(19) United States
(12) Patent Application Publication
(10) Pub. No.: US 2026/0284393 A1
(43) Pub. Date: Sep. 24, 2026

(54) NEUROMODULATION/NEUROSTIMULATION SYSTEM FOR RESTORING A MOTOR FUNCTION AND FACILITATING NEUROLOGICAL RECOVERY

(71) Applicants: ECOLE POLYTECHNIQUE FÉDÉRALE DE LAUSANNE, Lausanne (CH); COMMISSARIAT À L'ENERGIE ATOMIQUE ET AUX ENERGIES ALTERNATIVES, Paris (FR)

(72) Inventors: Henri LORACH, Paudex (CH); Andrea GALVEZ-SOLANO, Lausanne (CH); Grègoire COURTINE, Lausanne (CH); Jocelyne BLOCH, Paudex (CH); Guillaume CHARVET, Grenoble cedex 09 (FR); Fabien SAUTER-STARACE, Grenoble cedex 09 (FR); Tetiana AKSENOVA, Grenoble cedex 09 (FR)

(21) Appl. No.: 19/479,586

(22) PCT Filed: Apr. 26, 2024
(86) PCT No.: PCT/EP2024/061666

§ 371 Date: Oct. 28, 2025

(30) Foreign Application Priority Data
Apr. 28, 2023 (EP) 23170556.7

(51) Int. Cl. A61N 1/36; A61N 1/05; A61N 1/372; A61N 1/378

(52) U.S. Cl. CPC A61N 1/36062; A61N 1/0553; A61N 1/36146; A61N 1/37229; A61N 1/3787

(57) ABSTRACT

A neuromodulation/neurostimulation system for restoring a motor function of upper and/or lower limbs and facilitating neurological recovery in a patient with spinal cord injury, comprising: a processing unit configured to provide stimulation commands; a stimulation unit configured to deliver neuromodulation therapy, in particular epidural electrical stimulation, to dorsal roots of the spinal cord; a pulse generator operatively connected to the stimulation unit; and an implantable neurosensor including electronic components configured to detect electrocorticographic signals from the sensorimotor cortex of said patient, denoting a motor intention, wherein the processing unit, the stimulation unit and the neurosensor are operatively connected to define a brain-spinal interface, and wherein the processing unit is configured to collect and decode the electrocorticographic signals from the sensorimotor cortex into a corresponding motor intention of said patient; convert the decoded motor intentions into corresponding stimulation commands, and forward the stimulation command to the at least one pulse generator.

### CROSS REFERENCE TO RELATED APPLICATIONS

The present application is a U.S. National Phase of International Application No. PCT/EP2024/061666 entitled “NEUROMODULATION/NEUROSTIMULATION SYSTEM FOR RESTORING A MOTOR FUNCTION AND FACILITATING NEUROLOGICAL RECOVERY”, and filed on Apr. 26, 2024. International Application No. PCT/EP2024/061666 claims priority to European Patent Application No. 23170556.7 filed on Apr. 28, 2023. The entire contents of each of the above-listed applications are hereby incorporated by reference for all purposes.

### TECHNICAL FIELD

The present invention belongs to the technical field of spinal cord stimulation for restoring a motor function in a patient with spinal cord injury (SCI) or other neurological disorders such as stroke, Parkinson's disease, and/or other neurodegenerative disorders.

### BACKGROUND AND SUMMARY

More specifically, the present invention provides a neuromodulation/neurostimulation system for restoring a motor function of the upper and/or lower limbs and facilitating neurological recovery in a patient with SCI or other neurological disorders.

In order to perform a motor function, the brain delivers executive commands to the neurons located in the spinal cord. For instance, to perform a motor function of the lower limbs such as walking, the brain delivers executive commands to the neurons located in the lumbosacral spinal cord [1]. To perform a motor function of the upper limbs such as reach and grasp, the brain delivers executive commands to the neurons located in the cervical spinal cord [1].

While the majority of spinal cord injuries do not directly damage these neurons, the disruption of descending pathways interrupts the brain-derived commands that are necessary for these neurons to produce a motor function, e.g. walking, reach and grasp [2].

The consequence is therefore permanent paralysis.

It has been observed that epidural electrical stimulation (EES) targeting the individual dorsal root entry zones of the lumbosacral spinal cord enables the modulation of specific leg motor pools [3]-[6]. Similarly, EES targeting the cervical dorsal roots elicit upper limb muscle activity [56], [57].

The physiological principles underlying targeted EES of the spinal cord have presently been validated in 9 out of 9 treated subjects with incomplete [7] or complete [8] spinal cord injuries [26].

In particular, EES recruits large-diameter afferent fibers where they enter the spinal cord through the dorsal root entry, thereby activating motor neurons both directly and indirectly, contributing to transforming spinal circuits from a dormant to a highly excitable state.

The recruiting of these dorsal root entry zones with preprogrammed spatiotemporal sequences replicates the physiological activation of leg motor pools underlying motor functions such as standing and/or walking [2], [7]-[10].

The delivery of epidural electrical stimulation over the lumbar spinal cord enabled many individuals with spinal cord injury to regain adaptive control over the activity of otherwise paralyzed muscles.

This recovery has been documented in various independent studies, involving participants with complete sensorimotor paralysis [8], [26], [28]-[32].

These observations indicate that anatomically intact, yet functionally silent pathways from the brain can modulate the impact of EES on the activity of the spinal cord below the injury.

Clinical trials and behavioral experiments in human subjects revealed that EES boosts residual signals from the brain, which enables the modulation of paralyzed muscles.

However, this requires precise synchronization between the regions activated by residual commands from the brain and the location over which EES is applied.

Hence, it is important that the timing of multiple EES waveforms coincides with the intended movement of the limbs, e.g. legs, while a complex motor activity, e.g. walking, is carried out to synchronize the stimulation.

Therefore, the detection of an intended movement of the upper and/or lower limbs is critical in order to synchronize the delivered stimulation.

In recent studies, clickers have been used, allowing patients to trigger stimulation sequences.

As an alternative, motion sensors have been employed to detect the intended movements of the subject from body parts that have not been completely paralyzed by SCI or other neurological disorders.

For example, several patients learned to lean their body forward to raise the heel, and thus trigger a sequence of EES eliciting a swing phase.

Although this approach has proven sufficient to detect ongoing movements, nevertheless it failed to anticipate task transitions or the desire to implement volitional adjustment of movements, which are necessary to support mobility in everyday life.

This becomes even more critical when targeting the cervical spinal cord to restore upper limb movements.

Summarizing, clinical trials revealed that complex motor control for both the lower limb and the upper limbs cannot be achieved when relying exclusively on external sensors.

Furthermore, while using clickers and/or motor sensors, the control of a motor function, e.g. walking, was not perceived as completely natural by the patients.

Still further, in this approach, the patients showed limited ability to adapt leg movements to changing terrains and volitional demands.

Therefore, there is a need for an improved solution that allows to restore and control a motor function of the upper and/or lower limbs in patients with SCI or other neurological disorders in a way that is perceived as completely natural.

It is an object of the present invention to provide a neuromodulation/neurostimulation system that allows enabling volitional control over the timing and amplitude of muscle activity, thereby restoring a more natural and adaptive control of a motor function, such as reaching and/or grasping and/or standing and/or walking, in a patient with SCI or other neurological disorders.

The present invention provides a neuromodulation/neurostimulation system for restoring a motor function of the upper and/or lower limbs and facilitating neurological recovery in a patient with spinal cord injury (SCI) and/or other neurological disorders.

In particular, the neuromodulation/neurostimulation system according to the invention comprises:
a processing unit, which is configured and adapted to provide stimulation commands;
an implantable and/or external stimulation unit, which is configured and adapted to deliver neuromodulation, in particular epidural electrical stimulation (EES) to the dorsal roots of the spinal cord of said patient;
at least one implantable or external pulse generator, operatively connected to the stimulation unit;
an implantable neurosensor including electronic components that are configured and adapted to detect electrocorticographic (ECOG) signals from the sensorimotor cortex of said patient, denoting a motor intention of said patient,
wherein the processing unit, the stimulation unit and the implantable neurosensor are operatively connected to define a brain-spinal interface, and
wherein the processing unit is configured and adapted to:
collect and decode, preferably in real-time, the ECOG signals from the sensorimotor cortex into a corresponding motor intention of said patient;
converting, preferably in real-time, the decoded motor intentions into corresponding stimulation commands, and
forwarding, preferably in real-time, the stimulation command to the at least one IPG.

The present invention relates to a system for providing neuromodulation/neurostimulation to the spinal cord of a patient, in particular to elicit a motor function of the patient.

In particular, the invention provides a neuromodulation/neurostimulation system for restoring a motor function of the upper and/or lower limbs and facilitating neurological recovery in a patient with spinal cord injury and/or other neurological disorders.

The system comprises a processing unit, which is configured and adapted to provide stimulation commands.

For example, the processing unit may comprise a laptop and a base station.

Alternatively, the processing unit may be embedded on a mobile device, such as a smartphone, tablet computer or the like.

Also, as a further alternative, the processing unit may be directly embedded in the implantable neurosensor.

The system further comprises a stimulation unit.

For instance, said stimulation unit can be implantable.

Alternatively, said stimulation unit can be external.

The stimulation unit is configured and adapted to deliver neuromodulation therapy.

In particular, the stimulation unit can be configured and adapted to deliver epidural electrical stimulation (EES) to the dorsal roots of the spinal cord of said patient.

However, the invention is not limited to a configuration where the stimulation unit is configured and adapted to deliver EES.

For instance, the stimulation unit may as well be configured and adapted to deliver functional electrical stimulation (FES).

Delivery of FES may be convenient in order to restore/control a function of the upper limbs.

For instance, delivery of FES to or close to a hand of the patient has proven effective for the purpose of restoring hand movement of a patient.

Still further, the system comprises at least one pulse generator, operatively connected to the stimulation unit.

The pulse generator may be an implantable pulse generator (IPG).

Alternative, the pulse generator can be external.

There is also an implantable neurosensor, including electronic components that are configured and adapted to detect electrocorticographic (ECOG) signals from the sensorimotor cortex of said patient, denoting a motor intention of said patient.

The processing unit, the stimulation unit and the implantable neurosensor are operatively connected to define a brain-spinal interface (BSI).

Advantageously, the processing unit, the implantable stimulation unit and the implantable neurosensor may be operatively connected through a wireless connection.

The processing unit is configured and adapted to:
collecting and decoding, preferably in real-time, the detected ECoG signals from the sensorimotor cortex into a corresponding motor intention of said patient;
converting, preferably in real-time, the decoded motor intentions into corresponding stimulation commands, and
forwarding, preferably in real-time, the stimulation command to the at least one pulse generator, e.g. an IPG.

The invention is based on the basic idea that, by establishing a direct link between ECOG signals denoting a motor intention of a patient and the analog modulation of neurostimulation, e.g. EES, targeting the spinal cord regions involved in the intended motor function, the patient is enabled to experience a natural control of the performed motor function, e.g. reaching and/or grasping and/or standing and/or walking.

Real-time as used here as a term is to be understood such that real-time is a guaranteed level of computer responsiveness within a specified time constraint, usually milliseconds or microseconds, between an event and its response deadline. Real-time describes a human sense of time (rather than machine time) that seems immediate, see also DIN ISO/IEC 2382 and DIN 44300.

Otherwise stated, the digital bridge [9], [11]-[15] defined between the brain and the spinal cord allows enabling volitional control over the timing and amplitude of a muscular activity, restoring a more natural and adaptive control of a motor function, e.g. standing or walking, reach and grasp in a patient with SCI or other neurological disorders.

Residual and prosthetic pathways converge on the same neurons below the injury, thereby enabling a graded and sustained control over the activity of muscles.

This cooperation plays an essential role in the reorganization of neuronal pathways that mediates neurological recovery in response to neurorehabilitation performed by using the system according to the present invention.

Comparable observations were already reported in the context of clinical trials where electroencephalographic signals were coupled to an exoskeleton or functional electrical stimulation (FES) of muscles during gait rehabilitation in SCI patients [19], [21], [33].

However, the poor quality of electroencephalographic signals in mobile conditions combined with the impracticality of this technological framework determined an impediment to the clinical implementation of these non-invasive strategies.

Neurorehabilitation performed through the system according to the present invention has also proven effective in improving neurological recovery.

A clinical trial on a SCI patient (in detail, cf. section “Clinical trial on a human subject” below) showed that, after forty sessions of rehabilitation treatment using the brain-spine interface, the patient regained the ability to walk with crutches overground even when stimulation was switched off.

Although focused on one muscle group (i.e. hip muscles), this neurological recovery translated into the ability to lift the leg against gravity without stimulation. This recovery supported independent walking with crutches.

In preclinical models, neurorehabilitation supported by a digital bridge between the brain and the spinal cord triggered superior recovery when compared to EES alone [11].

Brain-controlled neuromuscular stimulation also mediated durable functional improvements of the engaged muscles after stroke and spinal cord injury [19], [21], [35].

The patient involved in the clinical trial previously reached a plateau of recovery after intensive rehabilitation using EES alone in the course of a previous clinical trial.

Subsequent neurorehabilitation performed through the system according to the invention triggered a reorganization of neuronal pathways that was responsible for the additional neurological recovery.

These results suggest that establishing a continuous link between the brain and spinal cord promotes the reorganization of residual neuronal pathways that link these two regions under normal physiological conditions [36]-[39].

The effective amount of neurological recovery remains however correlated to the severity of the lesion.

Although the clinical trial was focused on restoring a motor function of the lower limbs, in particular walking, the same concept may as well be used for restoring arm and/or hand movements after spinal cord injury [40] and stroke [41], by delivering neuromodulation therapy, e.g. EES or FES to the cervical region of the spinal cord.

Advantageously, the processing unit includes software means that are configured and adapted to decode one or more brain signal from the received ECOG signals.

As mentioned, the stimulation unit may be configured and adapted to deliver neuromodulation therapy, e.g. EES to, the lumbosacral region of the spinal cord of the patient.

This allows to restore a motor function of the lower limbs, such as control over independent joint movements, in particular hip, knee and ankles bilaterally, performing leg press exercises or other muscle strengthening exercises, standing, walking on a plain terrain, walking on a complex terrain, and/or stairs climbing.

In particular, the stimulation unit may be configured and adapted to deliver neuromodulation therapy, e.g. EES to spinal cord segments from L1 to S2.

Alternatively, the stimulation unit may be configured to deliver neuromodulation therapy, e.g. EES, to the cervical region of the spinal cord of the patient.

In particular, the stimulation unit may be configured and adapted to deliver neuromodulation therapy, e.g. EES, to spinal cord segments from C4 to T1.

This allows to restore a motor function of the upper limbs, such as independent control over the different joint movements, in particular shoulder rotation, shoulder flexion/extension, shoulder adduction/abduction, elbow flexion/extension, pronation/supination, and/or hand opening and closing, enabling to perform functional tasks such as reaching and/or grasping, self-feeding, self-care, transfers, and/or object manipulation.

Advantageously, the implantable neurosensor may be configured to be located over the dura mater above a fraction of the sensorimotor cortex.

In particular, the most active part of the sensorimotor cortices is identified by fMRI and MEG prior to surgery.

Therefore, the implantable neurosensor may be implanted without the need to open the dura mater, which significantly reduces the invasiveness of the system.

The implantable neurosensor may include at least one cortical implant comprising 64 electrodes that are configured to be epidurally implanted over the dura mater above a fraction of the sensorimotor cortex.

For instance, the implantable neurosensor may include a single cortical implant.

Alternatively, the implantable neurosensor may include two cortical implants.

Preferably, the 64 electrodes of each cortical implant may be arranged according to an 8-by-8 grid.

Even more preferably, said 8-by-8 grid has a 4 mm×4.5 mm pitch in the antero-posterior and medio-lateral axes, respectively.

Advantageously, the electronic components of the implantable neurosensor may be provided within a case having the same thickness of the patient's skull.

Preferably, the case may have a circular shape.

Even more preferably, the case may have a circular shape having a diameter of 50 mm.

Advantageously, the case can be made of Titanium.

This particular geometry of the cortical implants enables close and stable contacts between the electrodes and the dura mater, while rendering the neuro sensor invisible once implanted within the skull.

The cortical implants have structural and functional features that are similar to that of known WIMAGINE® implants [16], [17].

In particular, the WIMAGINE® technology provides a wireless 64-channel ElectroCorticoGram (ECoG) recording implant for clinical applications, said device being aimed at interfacing a cortical electrode array to an external computer for neural recording and control applications. This active implantable medical device is able to record neural activity on 64 electrodes with selectable gain and sampling frequency, with less than 1 μV (RMS), preferably less than 0.7 μVRMS input referred noise in the [0.5 Hz-300] Hz band. The implant is powered remotely through an inductive link at 13.56 MHz, which provides up to 100 mW (30 mA at 3.3V). The digitized data is transmitted wirelessly to a custom designed base station, connected to a PC. The hermetic housing and the antennae are designed and optimized to ease surgery [52]-[55].

In particular, in the WIMAGINE® technology, the implant is configured to communicate wirelessly on the Medical Implant Communication Service (MICS) band at 402-405 MHz with a custom-designed base station connected to a PC, and complies with the regulations applicable to class III AIMD [53].

The WIMAGINE® technology is based on a custom integrated circuit (ASIC) for signal conditioning, amplification and digitization and also on commercial components for RF transmission. The technology may support the RF transmission of a 32-channel EEG recording sampled at 600 Hz or 1 kHz with a 12-bit resolution [55]. The technology may also operate at 450 kb/s in 4-FSK mode, to allow recording of the 32 channels at 1 kHz. The system may further include a first external, high frequency antenna which is configured and adapted to power the implanted components of the system through inductive coupling (HF, 13.56 MHz).

There is also a second external, ultra-high frequency antenna (UHF, 402-405 MHz), which is configured and adapted for real-time transfer of the detected ECOG signals to the processing unit.

Preferably, the first external, high frequency antenna and the second external, ultra-high-frequency antenna may be embedded within a wearable device.

Advantageously, said wearable device can be a head-mounted device, e.g. a headset.

This solution ensures a reliable coupling with the cortical implants.

Advantageously, the first external, high frequency antenna and the second external, ultra-high frequency antenna may be operatively connected to a base station.

In particular, said base station may be configured and adapted to perform at least data conditioning, power generation, and/or synchronization with other devices to allow interfacing with the processing unit.

The processing unit may be embedded within a wearable device, that can be worn by the patient (e.g. at the wrist or elbow or belt, or carried in a backpack).

Alternatively, the processing unit may be carried on a walking-assistance device.

Therefore, the patient is able to conveniently carry this unit without inconvenience or otherwise feeling uncomfortable.

The pulse generator, e.g. an IPG, may be implemented with at least one communication module that is configured and adapted to implement real-time adjustment over the stimulation target and timing of delivered neuromodulation therapy, e.g. EES, with a latency of about 100 ms.

Accordingly, the effectiveness and sensitivity of delivered stimulation can be significantly improved.

The stimulation unit may include implantable paddle lead comprising an array of 16 electrodes configured to be epidurally implanted at a target location of the spinal cord of the patient.

In particular, said stimulation may include at least stimulation frequency, stimulation amplitude, pulse width, location of one or multiple cathodes, and/or location of one or multiple anodes.

For instance, the implantable stimulation unit may be configured to deliver EES with stimulation frequency between 10 Hz and 150 Hz.

Additionally or alternatively, the stimulation unit may be configured to deliver EES with pulse width between 200 us and 1000 us.

Additionally or alternatively, the stimulation unit may be configured to deliver EES with stimulation amplitude between 0 mA and 25 mA.

Any combinations of cathodes and anodes may be configured to access the desired spinal segments from the generated electric field.

The present invention also provides a walking-assistance device.

In particular, said walking-assistance device is configured and adapted for use with the above-described neuromodulation/neurostimulation system.

The walking-assistance device provides assistance for the patient during locomotion and further improves the overall safety conditions during use.

The walking-assistance device includes at least one charging module connected to a power inlet.

This allows for performing a charging operation while the device is not in use.

The walking-assistance device further includes a user interface, which allows the user to trigger, stop and/or adjust delivered stimulation.

Therefore, the patient is enabled to control the delivered stimulation in an intuitive, user-friendly manner.

The processing unit of the neuromodulation/neurostimulation system may be embedded within the walking-assistance device.

Optionally, the walking-assistance device may include additional components such as a camera for markerless tracking of body posture, an Inertial Measurement Unit (IMU) base station, one or more manual clickers, and/or one or more hybrid clickers.

Overall, the walking-assistance device in combination with the above-described neuromodulation/neurostimulation system facilitates the clinical implementation of neuromodulation therapy, e.g. EES, to restore a locomotion function, e.g. walking.

The neuromodulation/neurostimulation system according to the invention may be used in a method for restoring a motor function of the upper and/or lower limbs and facilitating neurological recovery in a patient with SCI and/or other neurological disorders.

In particular, such an exemplary method may include the following steps of:
providing a processing unit, which is configured and adapted to provide stimulation commands;
providing an implantable or external stimulation unit, which is configured and adapted to deliver neuromodulation therapy, in particular EES to the dorsal roots of the spinal cord of said patient;
providing at least one implantable or external pulse generator, operatively connected to the stimulation unit, and
providing an implantable neurosensor including electronic components that are configured and adapted to detect electrocorticographic (EcoG) signals from the sensorimotor cortex of said patient, denoting a motor intention of said patient,
the processing unit, the stimulation unit and the implantable neurosensor being operatively connected to define a brain-spinal interface,
collecting and decoding, preferably in real-time, through said processing unit, the EcoG signals from the sensorimotor cortex into a corresponding motor intention of said patient;
converting, preferably in real-time, through said processing unit, the decoded motor intentions into corresponding stimulation commands, and
forwarding, preferably in real-time, through said processing unit, the stimulation command to the at least one pulse generator.

### BRIEF DESCRIPTION OF THE FIGURES

Further details and advantages of the present invention shall now be disclosed in connection with the drawings, where:

FIG. 1 is a schematic view of a neuromodulation/neurostimulation system for restoring a motor function in the upper and/or lower limbs and facilitating neurological recovery in a mammal, in particular a human, with spinal cord injury (SCI) or other neurological disorder, according to an embodiment of the present invention;

FIG. 2 is a block diagram illustrating the neuromodulation/neurostimulation system according to the invention, in greater detail;

FIG. 3 is a block diagram illustrating a detail of the WIMAGINE® technology, implemented in the neuromodulation/neurostimulation system according to the invention. In the Figure, dashed lines are used to denote wireless connections, while solid lines are used to denote wired connections;

FIG. 4 is a picture showing the components of the neuromodulation/neurostimulation system according to the invention;

FIG. 5 is a diagram showing the different components and sub-components of the neuromodulation/neurostimulation system according to the invention;

FIGS. 6a-c are diagrams showing the design technology and implantation of the BSI. In detail: FIG. 6a. Two cortical implants composed of 64 electrodes are positioned epidurally over the sensorimotor cortex to collect ECoG signals. A processing unit predicts motor intentions and translates these predictions into the modulation of epidural electrical stimulation programs targeting the dorsal roots entry zones of the lumbosacral spinal cord. Stimulations are delivered by an implantable pulse generator connected to a 16-electrode paddle lead. FIG. 6b. Images reporting the pre-operative planning of cortical implant locations, and post-operative confirmation. L, left; R, right. FIG. 6c. Personalized, computational model predicting the optimal localization of paddle lead to target the dorsal root entry zones associated with lower muscles and post-operative confirmation;

FIGS. 7a-d are diagrams showing the technological and computational design underlying the BSI. In particular. FIG. 7a. Photographs reporting the geometry and features of the WIMAGINE® implant, including 64 platinum-iridium (90:10) electrodes with 4 mm×4.5 mm pitch (in antero-posterior and medio-lateral axes, respectively). Two external antennas are embedded within the implant. The first antenna powers the implanted electronics through inductive coupling at high frequency (HF, 13.56 MHz) while the second ultra-high frequency antenna (UHF, 402-405 MHz) transfers the recorded signals outside the body. FIG. 7b. Two external antennas embedded in a personalized 3D-printed headset power the implant and recover the streamed signals that are then transferred to a base station. The base station manages the powering of the implants, synchronization and conditioning of the raw data. FIG. 7c. A decoding pipeline computes temporal, spectral and spatial figures embedded in the ECOG signals related to the intention to move. These features are then uploaded into the decoding algorithm that decodes the attempts to move the lower limbs based on a tailored, recursive exponentially weighted Markov-switching multi-linear model algorithm [15]. This algorithm is a mixture of multilinear experts' algorithm integrating a Hidden Markov Model (HMM) classifier, called gating, and a set of independent regression models called experts. The gating classifier predicts the joint that is intended to be mobilized (i.e. hip, knee or ankle on each side) as well as the resting state, while each expert is dedicated to predicting the direction and relative amplitude of the intended movement. When updating is allowed, every 15 s, the coefficients of both linear regressions (β^{gate}, b^{gate}, β^{expert}, b^{expert} are updated through recursive partial least square along with the coefficients of the transition matrix T corresponding to the number of transitions between each state during this 15 s period (i.e. 150 new transitions). To support the production of standing and walking, the outputs of the model are encoded into updates of joint-specific stimulation programs that are constrained within pre-established functional ranges of amplitudes. FIG. 7d. A tailored, medical-grade software sends these updates to the implanted pulse generator through a chain of wireless communication systems, eventually delivering the stimulation through a paddle array implanted epidurally over the lumbosacral spinal cord.

FIGS. 8a-e are diagrams showing the calibration of the BSI. In particular: FIG. 8a. Identification of the spatial and spectral distributions of ECOG feature weights related to attempted left hip flexions. FIG. 8b. Calibration of anode/cathode configurations and stimulation parameters (frequency, range of amplitudes) to elicit left hip flexions including electromyographic signals from lower limb muscles. The polar plot reports the relative amplitude of muscle responses for the optimal configuration to target left hip flexors over the range of functional stimulation amplitudes (300 μs, 40 Hz, 14 to 16 mA). FIG. 8c. Online calibration of the BSI to enable volitional hip flexion in a seated position. Representative sequence reporting spectrogram, decoding probability and proportional modulation of stimulation amplitudes together with the resulting muscle activity and torque. The plot reports the convergence of the model over time, reaching 97+/−0.4% after 90 s. FIG. 8d. Similar representations after the calibration of the BSI to enable the control over hip, knee and ankle joints of the lower limbs. FIG. 8e. Confusion matrices reporting the decoding accuracy for each joint (74+/−7%, s.e.m.) and the accuracy of the stimulation for each targeted muscle group (83+/−6%, s.e.m.)

FIG. 9a-g are diagrams showing the calibration of the BSI. In particular. FIG. 9a. Post-operative localization of the cortical implants over the segmented brain of the participant that confirms the appropriate positioning of the 64-electrode grids over the activated regions of the primary motor cortex responding to attempted lower limb movements, as measured during functional magnetoencephalographic recordings. FIG. 9b. Post-operative localization of the paddle lead over the lumbosacral spinal cord to target lower limb muscles. FIG. 9c. Projection of linear regression weights associated with different lower limb movements (depicted on body schemes) onto the location of the implants, revealing the spatial segregation of movement-specific features. FIG. 9d. Electromyographic activity recorded from several lower limb muscles following a burst of epidural electrical stimulation using the more selective electrode configurations (schemes) and parameters (reported) translated into polar plots reporting the amplitude of muscle responses. FIG. 9e. Spatial distribution of linear regression weights associated with upper versus lower limb movements over the grid of 64 electrodes from each cortical implant. The firmware enabled the selection of a set of electrodes (e.g. 32) within the 64 electrodes of each implant. The red dots indicate the 32 selected electrodes from each implant based on the amount of identified movement-related information for each of the 64 electrodes. FIG. 9f. Spectral distribution of linear regression weights associated with upper versus lower limb movements, highlighting the importance of high sampling density in low frequencies compared to high frequencies. This ensemble of features guided the parameterization of the decoders. FIG. 9g. Detailed representation of the spatial and spectral repartition of weights associated with decoding of the 6 different lower limb joint movements;

FIGS. 10a-f are diagrams showing that BSI restores natural control of walking. In particular: FIG. 10a. Attempts to perform voluntary hip flexions without and with the BSI, including photos, the vertical elevation of the knee and hip flexor muscle activity. Bar plots report the mean values of these measurements. (n=3 attempts per condition, unpaired one-tailed t-test, ***, p<0.001). FIG. 10b. Chronophotography while walking with the BSI turned on, off, and then on again. Note the two decoded attempts that do not lead to muscle activity nor the execution of steps. FIG. 10c. Range of stimulation amplitude during walking. FIG. 10d. Bar plots reporting mean values of kinematic and muscle activity parameters during walking with the BSI turned off and on, (n=3 and 8 attempts for BSIOFF and BSION respectively, unpaired one-tailed t-test, ***, p<0.001, (p (iliopsoas activation)=3.4E-4, p (step height)=5.1E-10, p (hip angle)=2.7E-5, p (knee angle)=1.6E-9). FIG. 10e. Chronophotography of standing (voluntary pause) and walking with the BSI outdoors. The spectrogram, probabilities of left and right steps, and modulation of stimulation amplitudes illustrate the robustness of the performance and absence of false positive detections during a voluntary pause. FIG. 10f. Plots report the probability of right hip flexions over consecutive steps measured during the first session after the neurosurgical implantation (n=13 steps, accuracy=0.92+/−0.1 std, w=2.66 s+/−0.6 s std), and at two (n=46 steps, accuracy=0.93+/−0.1 std, w=2.64 s+/−0.6 s std), six (n=41 steps, accuracy=0.97+/−0.1 std, w=2.56 s+/−0.9 s std), and 11 months (n=29 steps, accuracy=0.97+/−0.1 std, w=1.71 s+/−0.4 s std) after the first activation of the BSI using updated models (see also FIG. 14);

FIGS. 11a-c are diagrams showing that the stability of the decoder enables safe utilization of the BSI. In particular. FIG. 11a. Chronophotography and associated spectrogram, probabilities of left and right steps, modulation of muscle activity, stimulation amplitudes, and peak probability of step cycles during a sequence involving walking, a voluntary pause (30 s, instructed), and resuming walking. The absence of false positive detections illustrates the robustness of the BSI. FIG. 11b. The bar plot reports the peak probability of walk (active) versus idle state, together with the confusion matrices reporting the detected rest versus left and right swing states (n=31 and n=49 samples for idle and active states respectively, unpaired one-tailed t-test ***, p<0.001). FIG. 11c. Photographs illustrating sit-to-stand capacities without and with the BSI, including bar plots reporting balance capacities (scores) measured using the Berg Balance Scale;

FIGS. 12a-h are diagrams showing neurological improvements following neurorehabilitation supported by the BSI in the absence of stimulation. In particular: FIG. 12a. Chronophotography illustrating the walking ability of the participant without any stimulation before enrolling in the previous clinical trial, after its completion, and after completion of the present clinical trial. FIG. 12b. Timeline of the two clinical trials, including a pie chart reporting the time during which the various types of neurorehabilitation exercises were practiced, as well as the home use of the BSI. FIG. 12c. Photographs showing the maximal hip flexion and associated flexor muscle activity before and after neurorehabilitation. FIG. 12d. Changes in lower limb motor scores over the course of both clinical trials. FIG. 12e. Plots reporting improvements in WISCI II scores over the course of both clinical trials. Neurorehabilitation supported by the BSI restored the capacity to walk over 10 m with crutches without any assistance nor stimulation. FIG. 12f-h. Plots reporting quantifications of the 6-minute walk test, weight-bearing capacity, time up and go, Berg Balance Scale and observational gait analysis (each dot refers to scores from a physiotherapist, (n=6, paired one-tailed t-test; **, p=0.002). N/A, not available;

FIGS. 13a-d are diagrams showing that the BSI normalizes gait parameters and supports walking on complex terrains. In particular: FIG. 13a. Principal component (PC) analysis applied on kinematic and muscle activity parameters during walking on a treadmill with stimulation alone versus BSI. During stimulation alone conditions, a closed-loop controller based on motion sensors attached to the lower limbs determines the parameters of stimulation. Each dot represents a gait cycle. The bar plot reports the Euclidean distance in the PC space between each sample and the centroid of the healthy steps. (n=119, n=30 and n=61 steps for healthy, EES only and BSI respectively, unpaired one-tailed t-test ***, p<0.001). Compared to stimulation alone, the BSI enabled walking with gait features that were closer to those quantified in healthy individuals. This similitude is highlighted in the bar plots, which report the mean values of kinematic parameters with a high factor loading on PC1. FIG. 13b. Quantitative measure of step length while walking with crutches. Steps below 10 cm are considered failed as illustrated in the stick plot diagram. EES only condition showed significantly shorter step length due to an increase of failed steps (n=26, n=43 for EES only and BSI respectively, Mann-Whitney U test one-tailed t-test ** p<0.01). FIG. 13c. Photographs illustrating walking capacities, together with bar plots that report quantifications of performances during the execution of various walking paradigms, including walking up and down a ramp, climbing stairs, and walking with crutches overground. FIG. 13d. Walking on changing terrains with obstacles and different textures (6 surfaces), as illustrated in the scheme on the left. Conventions are the same as in previous figures. Decoding stability is shown by overlayed probability curves of right hip flexions over consecutive steps (n=13 steps, Left accuracy=0.89+/−0.1 std, w=2.06 s+/−0.6 s std), and Left accuracy (n=13 steps, accuracy=0.91+/−0.1 std, w=2.06 s+/−0.4 s std);

FIGS. 14a-e are diagrams showing the long-term stability of the BSI supporting in particular walking. FIG. 14a. Recordings of the resting state were acquired regularly to evaluate the evolution of signal quality over time. Raw traces and power spectrum of an ECOG signal measured from a selected electrode are shown to illustrate the stability of the recorded signals. The plot reports the mean values of the power spectrum quantified over 2 minutes of resting state recorded at regular intervals over a period of nearly one year, showing a steady yet negligible decrease in signal quality over time (−0.03 dB/day). FIG. 14b. Plots reporting principal component analysis of gating coefficients from all the models used for supporting walking over the entire duration of the study. The size of each data point captures the relative time during which each model was used. FIG. 14c. Plots reporting the range of stimulation amplitudes and frequencies used over the entire course of the neurorehabilitation program, highlighting the robustness of the BSI over nearly six months of use. FIG. 14d. Spectrograms and decoding performance together with modulation of stimulation amplitude (relative) during self-paced walking enabled by the BSI. Plots report the probability of left and right hip flexion events (swing) measured over consecutive steps, and repeated at regular intervals over the entire time course of the clinical trial. FIG. 14e. Median spectrograms around the right hip flexion attempts during different time periods along training (n=100 attempts in each period). The average rectified modulations show a significant increase with time (n=64 electrodes, R2=0.68, p<0.001).

FIG. 15 is a diagram showing BSI supporting control over isolated lower limb movements. The same model was used to enable the participant to exert control over 6 joints from both sides during two sessions over 2 months. Conventions are the same as in previous figures;

FIGS. 16a-c are diagrams showing the design and configuration of the BSI for independent use at home FIG. 16a. An integrated walker was designed and fabricated to incorporate the different hardware composing the BSI, thereby maximizing the practicability of the technological platform for use at home. The system is battery-powered and can operate autonomously for approximately 2 hours without any supervision. FIG. 16b. Sequence showing the different steps to configure the BSI, including the positioning of the communication headset, uploading of a BSI program, monitoring of signal quality to ensure appropriate placement of the antennas, and adjusting the minimum and maximum amplitudes of the stimulation. The participant has been using the BSI independently to support neurorehabilitation and daily life activities for over nearly one year. Positioning the hardware and configuring the BSI requires approximately 5 minutes. FIG. 16c. Usage log and performance quantification of the participant after the main phase of the study as a cumulative number of decoded steps and cumulative time of use over a period of 181 days, i.e. since the participant returned to his home;

FIGS. 17a-c are diagrams referring to a clinical trial on a human patient, in particular showing implantation of a system according to the invention to restore upper limb function in said patient. FIG. 17a. Detailed planning and execution. FIG. 17b. Pre-operative planning of the WIMAGINE® implant location. Here, fMRI is employed for precise identification of motor cortical areas activated during arm and hand functional attempts. The far-right images confirm the post-operative implant location. L (Left), R (Right), M1 (Primary motor cortex), and S1 (Primary somatosensory cortex). FIG. 17c. Personalized computational model guiding the surgical planning of the cervical paddle lead location. This model incorporates factoring in cervical vertebral anatomy, location of the spinal cord lesion, and implanted instrumentation. In this participant, two leads were strategically implanted to cover the entire length of the “preserved” cervical enlargement, caudal to the injury site. The segmented images include the spinal cord lesion between C4 and C5. To prioritize examining the potential functional restoration of the participant's dominant hemisphere, cortical implant and leads were intentionally lateralized in this proof-of-concept trial. Post-operative confirmatory CT provides visual confirmation of the paddles which are intentionally overlapping to prevent potential dead stimulation spaces;

FIG. 18 is a diagram showing how electrical stimulation of the cervical dorsal roots triggers selective upper limb muscle activation, in the clinical trial of FIG. 17;

FIGS. 19a-e are diagrams showing decoding performance in the clinical trial of FIG. 17;

FIG. 20a-b are diagrams showing that delivery of stimulation to the cervical region enables reaching movements, in the clinical trial of FIG. 17. FIG. 20a. Decoding of elbow extension attempts are translated into EES triggering significant triceps activation (Left). When the stimulation is turned off, although attempts are requested, reaching cannot be achieved. FIG. 20b. Comparison between muscle activity (maximum of rectified emg envelope) as well as joint angle with and without use of the BSI, demonstrating significant increase of muscle activation and range of motion during movement attempts (p>0.001, unpaired t-test, n=5 repetitions);

FIG. 21 is a diagram showing experimental evidence referring to rehabilitation therapy (50 sessions) in the context of the clinical trial of FIG. 17, demonstrating that use of the system of the invention for upper limb movements triggers neurological and functional recovery in the participant. Here, the participant gained 8 points over the motor score of the treated arm.

### DETAILED DESCRIPTION

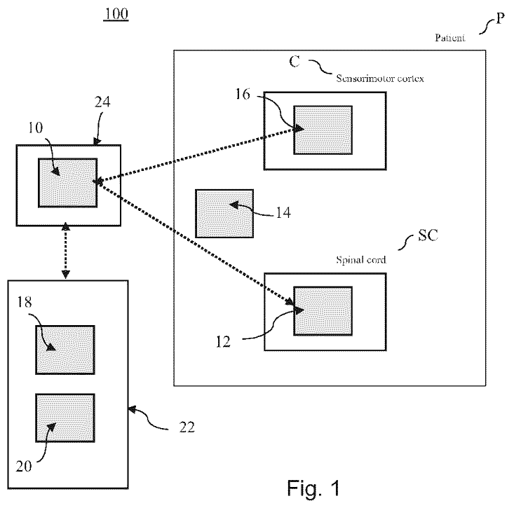

FIG. 1 provides a schematic overview of a neuromodulation/neurostimulation system 100 according to an embodiment of the present invention.

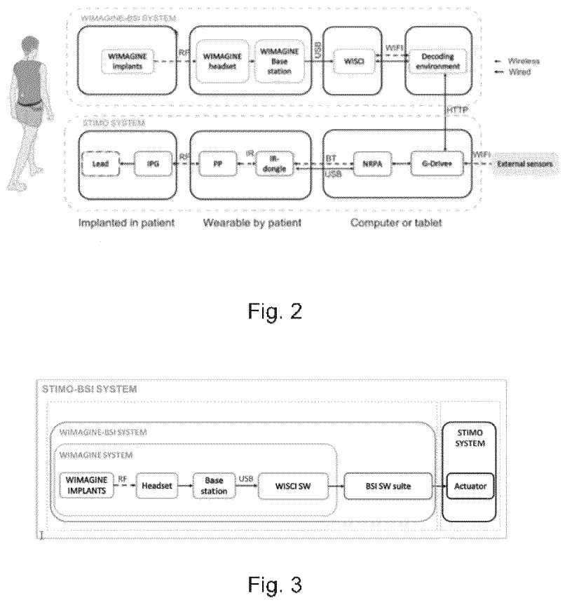

Further details of the neuromodulation/neurostimulation system 100 are provided in FIGS. 2-5.

In particular, the system 100 is configured and adapted for restoring the motor function of the upper and/or lower limbs and facilitating neurological recovery in a patient with SCI or other neurological disorders.

The system 100 comprises a processing unit 10, which is configured and adapted to provide stimulation commands.

The system 100 further includes a stimulation unit 12.

In the present embodiment, the stimulation unit 12 is implantable.

However, the stimulation unit 12 may as well be external.

In the present embodiment, the stimulation unit 12 is in particular configured and adapted to deliver epidural electrical stimulation (EES) to the dorsal roots of the spinal cord SC of said patient P.

However, the invention is not limited to a configuration where the stimulation unit 12 is configured and adapted to deliver EES.

As a non-limiting example, the stimulation unit 12 may as well be configured and adapted to deliver functional electrical stimulation (FES).

Still further, the system 100 comprises at least one pulse generator (IPG) 14, operatively connected to the stimulation unit 12.

In the present embodiment, the pulse generator is an implantable pulse generator (IPG) 14.

However, the pulse generator 14 may as well be external.

Also, the system 100 comprises an implantable neurosensor 16 including electronic components that are configured and adapted to detect electrocorticographic (ECOG) signals from the sensorimotor cortex C of said patient P, denoting a motor intention of said patient P.

The processing unit 10, the stimulation unit 12 and the implantable neurosensor 16 are operatively connected to define a brain-spinal interface (BSI).

In the shown embodiment the processing unit 10, the stimulation unit 12, and the implantable neurosensor 16 are operatively connected through a wireless connection (FIG. 1).

The processing unit 10 is configured and adapted to:
collect and decode, preferably in real-time, the ECoG signals from the sensorimotor cortex C into a corresponding motor intention of said patient P;
converting, preferably in real-time, the decoded motor intentions into corresponding stimulation commands, and
forwarding, preferably in real-time, the stimulation command to the at least one IPG 14.

In the present embodiment, the processing unit includes software means that are configured and adapted to decode one or more brain signal from the received ECOG signals.

For example, the processing unit 10 may comprise a laptop and a base station.

Alternatively, the processing unit 10 may be embedded on a mobile device, such as a smartphone, tablet computer or the like.

Also, as further alternative, the processing unit 10 may be directly embedded in the implantable neurosensor 16.

In the present embodiment, the stimulation unit 12 is configured and adapted to deliver EES to the lumbosacral region of the spinal cord SC of said patient P to restore a motor function of the lower limbs.

In particular, said motor functions of the lower limbs may include control over independent joint movements, in particular hip, knee and ankles bilaterally, standing, walking on a plain terrain, walking on a complex terrain, and/or stairs climbing.

In the present embodiment, the stimulation unit 12 is configured and adapted to deliver EES to spinal cord segments from L1 to S2.

Alternatively, the stimulation unit 12 may also be configured and adapted to deliver EES to the cervical region of the spinal cord SC of said patient P to restore a motor function of the upper limbs.

Preferably, the stimulation unit 12 may be configured to deliver EES to spinal cord segments from C4 to T1.

In particular, said motor functions of the upper limbs may include independent control over the different joint movements, in particular. shoulder rotation, shoulder flexion/extension, shoulder adduction/abduction, elbow flexion/extension, pronation/supination, hand opening and closing, enabling to perform functional tasks such as reaching and/or grasping, self-feeding, self-care, transfers, and/or object manipulation.

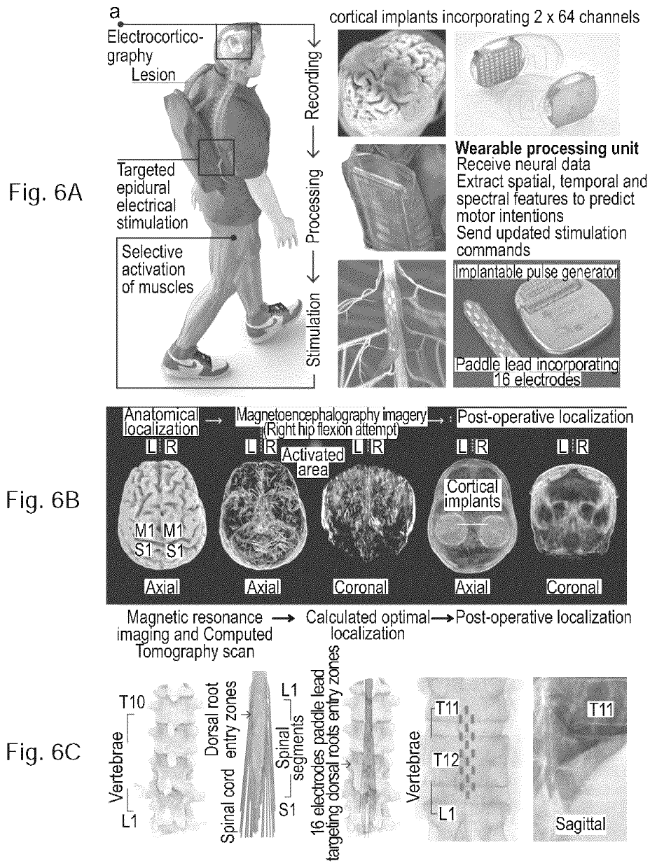

In the present embodiment, the implantable neurosensor 16 is configured to be located over the dura mater above a fraction of the sensorimotor cortex C, as shown in FIG. 6a.

In the present embodiment, the implantable neurosensor 16 includes two cortical implants comprising 64 electrodes that are designed for epidural recording of a fraction of the sensorimotor cortex C.

Not shown is that, alternatively, the implantable neurosensor 16 may include a single cortical implant.

In particular, said 64 electrodes of each cortical implant are arranged according to an 8-by-8 grid, preferably an 8-by-8 grid that has a 4 mm×4.5 mm pitch in antero-posterior and medio-lateral axes, respectively.

The electronic components of the implantable neurosensor 16 are provided within a case having the same thickness of the patient's skull.

In particular, the case has a circular shape preferably with a diameter of 50 mm.

Advantageously, the case is made of Titanium.

In the shown embodiment, the system 100 further includes a first external, high frequency antenna 18, which is configured and adapted to power the implanted components of the system 100 through inductive coupling (HF, 13.56 MHz) (FIG. 1).

There is also a second external, ultra-high frequency antenna 20 (UHF, 402-405 MHz), which is configured and adapted for real-time transfer of the detected ECOG signals to the processing unit 10 (FIG. 1).

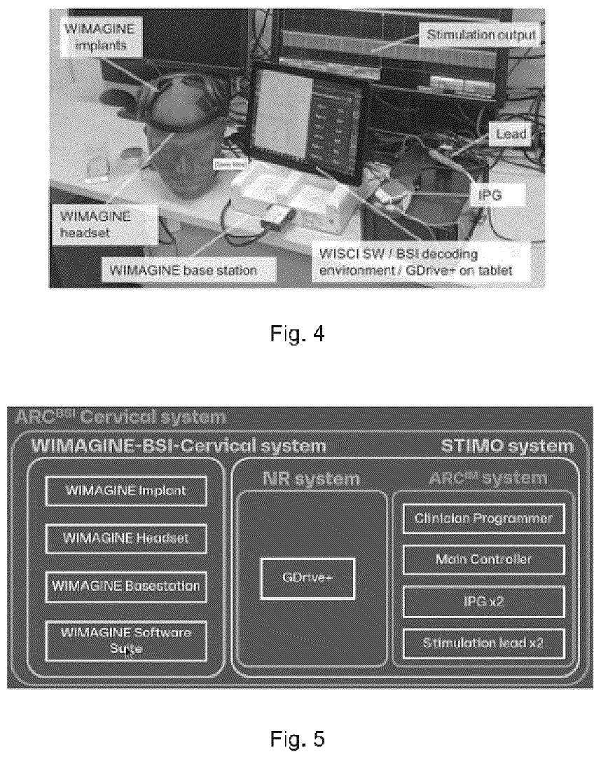

Advantageously, the first external, high frequency antenna 18 and the second external, ultra-high-frequency antenna 20 are embedded within a wearable device 22, in particular a head-mounted device 22, e.g. a headset (FIGS. 4, 6a, 7a-b).

In the present embodiment, the first external, high frequency antenna 18 and the second external, ultra-high frequency antenna 20 are operatively connected to a base station (FIGS. 2-5).

In particular, said base station is configured and adapted to perform at least data conditioning, power generation, and/or synchronization with other devices to allow interfacing with the processing unit 10.

The processing unit 10 is embedded within a wearable device 24 (FIG. 6a).

For example, the processing unit can be worn by the patient P on the wrist or elbow or carried in a backpack (FIG. 6a).

In the present embodiment, the IPG 14 is implemented with at least one communication module that is configured and adapted to implement real-time adjustment over the stimulation target and timing of delivered EES with a latency of about 100 ms.

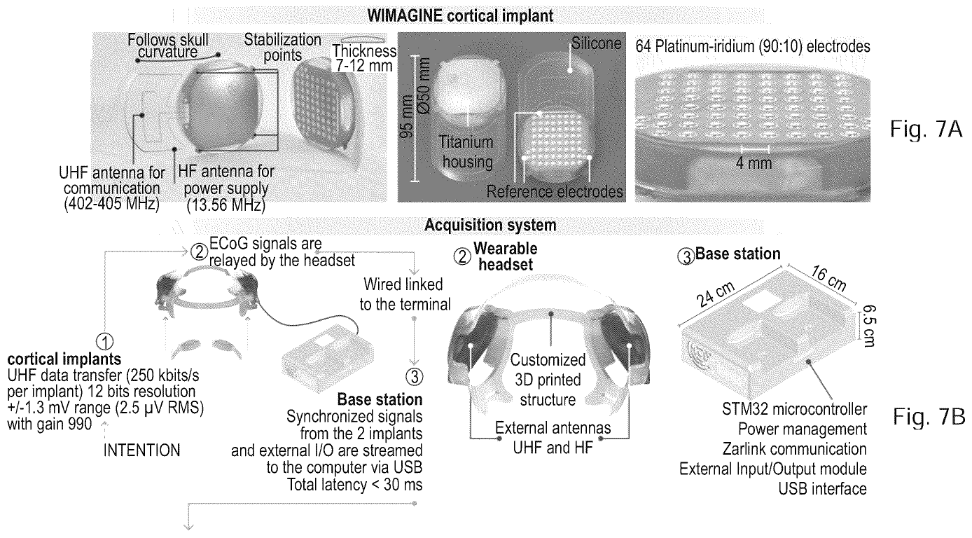

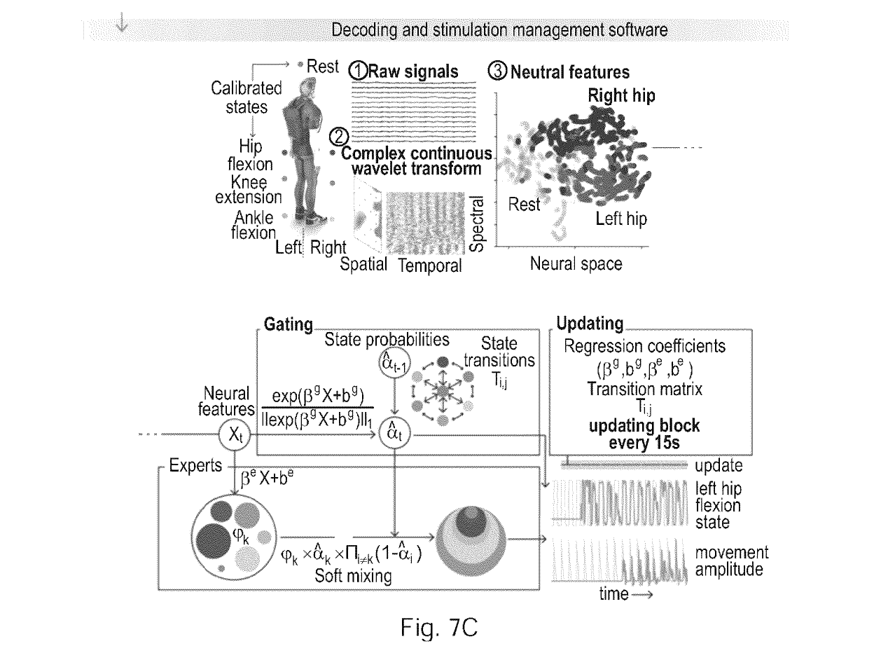

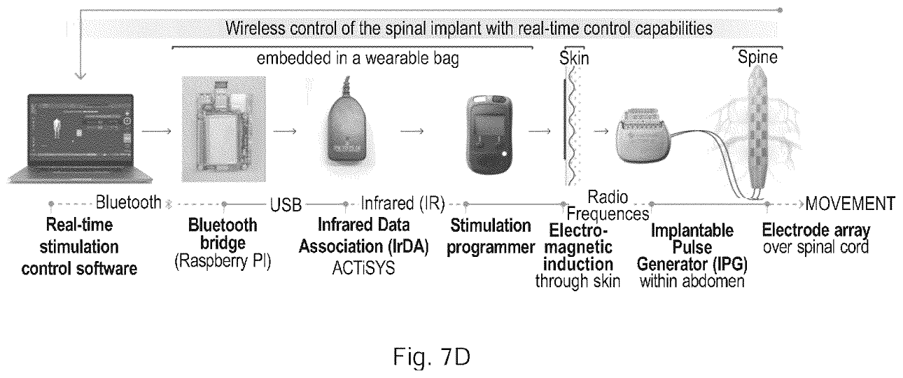

In the present embodiment, the stimulation unit 12 includes an implantable paddle lead comprising an array of 16 electrodes configured to be epidurally implanted at a target location of the spinal cord SC of said patient P (FIGS. 4, 7a).

The stimulation unit 12 is configured to deliver EES according to predefined stimulation parameters, wherein said predefined stimulation parameters include at least stimulation frequency, stimulation amplitude, pulse width, location of one or multiple cathodes and/or location of one or multiple anodes.

In the present embodiment, the stimulation unit 12 is configured to deliver EES with stimulation frequency between 10 Hz and 150 Hz.

Also, in the present embodiment, the stimulation unit 12 is configured to deliver EES with pulse width between 200 us and 1000 us.

Still further, in the present embodiment, the stimulation unit 12 is configured to deliver EES with stimulation amplitude between 0 mA and 25 mA.

Not shown is that the present invention further provides a walking-assistance device.

In particular, said walking-assistance device is configured for use with the neuromodulation/neurostimulation system 100 described above.

The walking-assistance device includes at least one charging module connected to a power inlet.

The walking-assistance device further includes a user interface allowing the user to trigger, stop and/or adjust delivered stimulation.

Advantageously, the processing unit 10 of the neuromodulation/neurostimulation system 100 may be embedded within the walking-assistance device.

Optionally, the walking-assistance device may as well include additional components such as a camera for markerless tracking of body posture, an Inertial Measurement Unit (IMU) base station, one or more manual clickers, and/or one or more hybrid clickers.

As mentioned, the neuromodulation/neurostimulation system 100 according to the invention can be used in a method for restoring a motor function of the upper and/or lower limbs and facilitating neurological recovery in a patient P with SCI and/or other neurological disorders, said method including the following steps of:
providing a processing unit 10, which is configured and adapted to provide stimulation commands;
providing an implantable or external stimulation unit 12, which is configured and adapted to deliver neuromodulation therapy, in particular EES to the dorsal roots of the spinal cord SC of said patient P;
providing at least one implantable or external pulse generator 14, operatively connected to the stimulation unit, and
providing an implantable neurosensor 16 including electronic components that are configured and adapted to detect electrocorticographic (ECOG) signals from the sensorimotor cortex C of said patient P, denoting a motor intention of said patient P,
the processing unit 10, the stimulation unit 12 and the implantable neurosensor 16 being operatively connected to define a brain-spinal interface (BSI),
collecting and decoding, preferably in real-time, through said processing unit 10, the ECoG signals from the sensorimotor cortex C into a corresponding motor intention of said patient P;
converting, preferably in real-time, through said processing unit 10, the decoded motor intentions into corresponding stimulation commands, and
forwarding, preferably in real-time, through said processing unit 10, the stimulation command to the at least one pulse generator 14.

### Clinical Trials on Two Human Subject

### Neurosurgical Implantation of the BSI to Restore Lower Limb Movements.

A 38-year-old male subject was enrolled in the present clinical trial (clinicaltrials.gov, NCT04632290, hereinafter “the present clinical trial”), who experienced an incomplete cervical (C5/C6) spinal cord injury during a biking accident 10 years prior to enrollment.

The subject already participated in a previous clinical trial (clinicaltrials.gov, NCT02936453, hereinafter “the previous clinical trial”), which involved a 5-month neurorehabilitation program supported by targeted epidural electrical stimulation of the spinal cord [7], [8].

This program allowed the participant to regain the ability to step with the help of a front-wheel walker.

Despite the continued use of the stimulation at home for approximately three years, he had reached a neurological recovery plateau that motivated him to enroll in the present trial.

To guide the implantation of the BSI, preoperative planning procedures were developed, allowing for optimisation of the positioning of the recording and stimulation implants over the brain and spinal cord.

The BSI requires the detection of neural features related to the intention to move the left and right lower limbs.

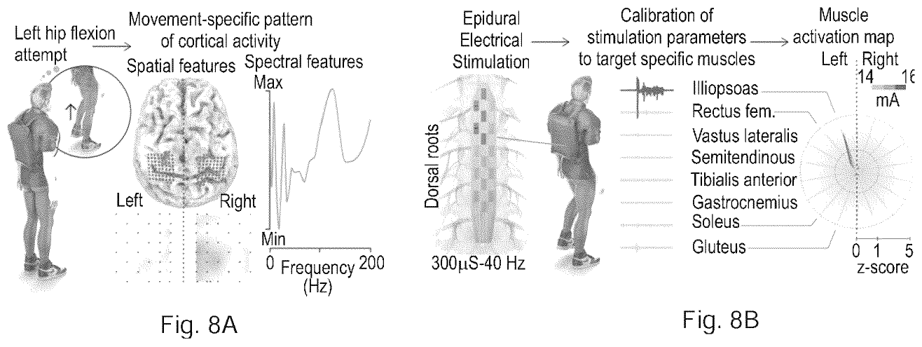

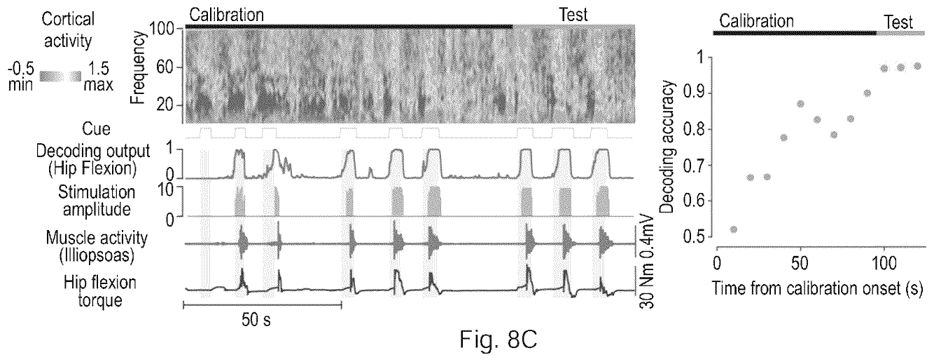

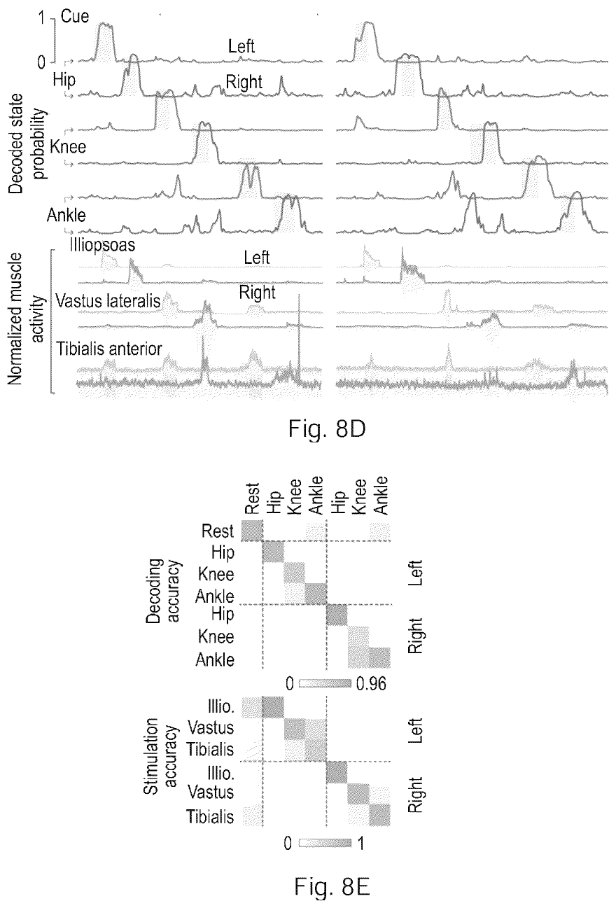

To identify the cortical regions most responsive to the attempt to move each joint of the lower limbs, anatomical and functional imaging data based on computerized tomography and magnetoencephalography were acquired (FIG. 8b)

These acquisitions identified the regions of the cerebral cortex that responded more robustly to the intention to move the left and right lower limbs.

This information was integrated with anatomical constraints to define the optimal positioning of the two ECOG recording implants that aim to decode left and right lower limb movements.

The location of both implants was uploaded onto a neuro-navigation system to establish the preoperative planning of the neurosurgical intervention.

Under general anaesthesia, a bi-coronal incision of the scalp was performed to enable two circular-shaped craniotomies over the planned locations of the left and right hemispheres using a tailor-made circular trephine that matched the diameter of the implants.

Then, the bone flaps were replaced with the two implantable recording devices before closing the scalp.

The paddle lead was positioned over the dorsal root entry zones of the lumbar spinal cord during the previous clinical trial.

The optimal position of the lead was identified using a personalized model of the spine elaborated from high-resolution structural imaging 8 (FIG. 6c).

The final location was optimized intraoperatively based on electrophysiological recordings [7], [8].

The implantable pulse generator, which was connected to the lead, was inserted into an abdominal subcutaneous pocket.

The patient was discharged 24 hours after each neurosurgical intervention.

### Configuration of Cortical and Spinal Implants

The calibration of the BSI required two prior independent procedures to select the features of ECOG recordings that discriminate the intention to move and to configure stimulation programs that modulate specific ensembles of lower limb muscles.

The first procedure consisted in extracting the spatial, spectral, and temporal features of ECOG signals that were linked to the intentions to mobilize each joint of both lower limbs.

For this purpose, the participant was requested to attempt hip, knee and ankle movements of the left and right sides in a seated position while ECOG signals were recorded concurrently.

This mapping enabled the identification of the electrodes, spectral features and

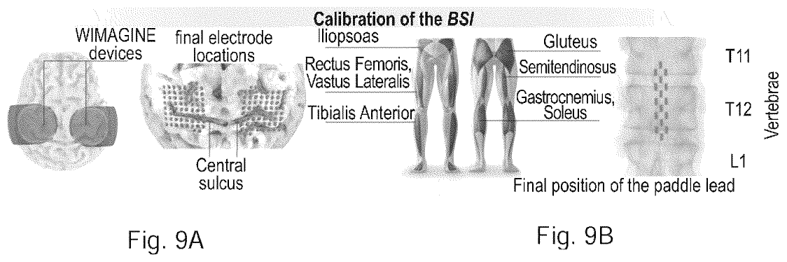

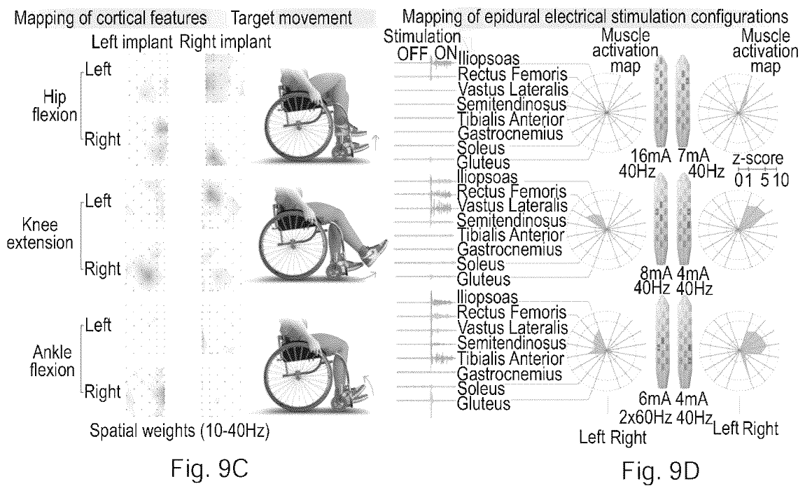

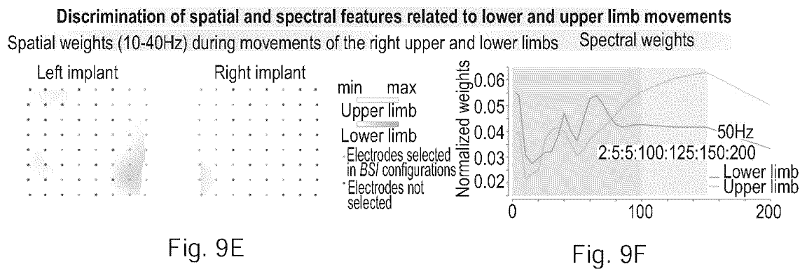

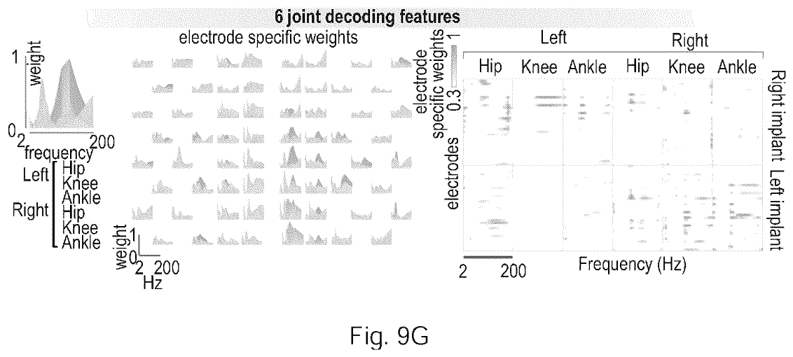

temporal windows that captured the larger amount of movement-related information [7], [18]-[21] (FIGS. 8a, 9).

The electrodes that measured neural signals correlating with leg movements were located on the most medial aspect of the implant, rostral to the central sulcus, as expected based on preoperative magnetoencephalographic recordings.

The spatial distribution of these electrodes followed a somatotopy that enabled the accurate discrimination of hip, knee and ankle movements (FIG. 9c).

Instead, upper limb-related movements coincided with the modulation of ECOG signals measured through electrodes located on the lateral aspect of the implant (FIG. 9f).

Movement-related information was contained over the entire range of beta and gamma frequency bands of ECOG signals (FIGS. 8a, 9g).

This procedure allowed us to configure the implants with the optimal features to enable the participant to operate the BSI (FIG. 9f-g).

The second procedure consisted in configuring stimulation programs (FIG. 8b).

Epidural electrical stimulation of the spinal cord can modulate specific ensembles of motor pools through the recruitment of the dorsal root entry zones projecting to the spinal cord regions wherein these motor pools reside [3], [22].

In turn, optimized configurations of anodes and cathodes can steer electric fields toward specific subsets of dorsal root entry zones to modulate well-defined ensembles of motor neuron pools [3], [8], [22].

This physiological principle enables the regulation of extension and flexion movements from each joint.

We leveraged this principle to configure a library of targeted epidural electrical stimulation programs that mobilized the hip, knee and ankle joints from both sides.

Concretely, combinations of anodes and cathodes, stimulation frequencies and amplitudes to steer electrical currents were configured in order to achieve gradual control over the activity of the targeted muscle groups (FIG. 9b-d).

### Adaptive Online Calibration of the BSI

Then the configurations of cortical and spinal implants were leveraged to calibrate the BSI based on a recursive, exponentially-weighted Markov-switching multilinear algorithm that linked ECoG signals to the control of epidural electrical stimulation parameters (FIG. 7).

The algorithm was designed to generate two separate predictions.

First, a gating model calculated the probability of the intention to move a specific joint.

Second, an independent multilinear model predicted the amplitude and direction of the intended movement.

The adaptive properties of the algorithm enabled online, incremental parametrization of the models throughout the period of calibration.

A hidden Markov model ensured the stability and robustness of predictions [23].

Then the predictions of the algorithm were translated into an analog controller that adjusted the amplitude of joint-specific stimulation commands.

These updated commands were delivered to the implantable pulse generator every 300 ms.

As early as the first session after the neurosurgical intervention, the algorithm calibrated a BSI that enabled the participant to control the relative flexion of the left and right hips of an avatar projected on a screen.

Then the analog control was integrated over the stimulation amplitude to the algorithm.

From a lying position, within less than two minutes the participant was able to control the activity of hip muscles to generate torque with an accuracy of 97% (FIG. 8c).

Then, this BSI framework was expanded to enable the participant to control the relative amplitude of hip, knee and ankle joints bilaterally along with the resting state.

Using this proportional BSI combining 7 states, the participant achieved gradual control over the movement of each joint bilaterally with an accuracy of 74+/−7%, whereas the chance level was limited to 14% (FIG. 8d-e).

The latency of the decoder was as low as 1.1 s (+/−0.15 s s.e.m.) for the 7 states.

These early sessions validated the procedure for the rapid, robust and accurate calibration of a BSI operating over multiple dimensions.

### Immediate Recovery of Natural Walking

We next asked whether this procedure supports the calibration of a BSI that restores natural control of walking.

Walking involves well-defined sequences of muscle activation patterns that support weight acceptance, propulsion and swing of the left and right lower limbs.

These sequences coincide with the activation of motor pools located within well-segregated regions of the lumbosacral spinal cord [7], [24].

Therefore, the stimulation programs have been selected within the library that targeted muscles associated with weight acceptance, propulsion and swing functions, and then linked to decoding probabilities.

BSI was calibrated to enable the participant to control the relative amplitude of stimulation programs for weight acceptance and swing functions.

First, this BSI was tested during voluntary elevations of the foot while standing.

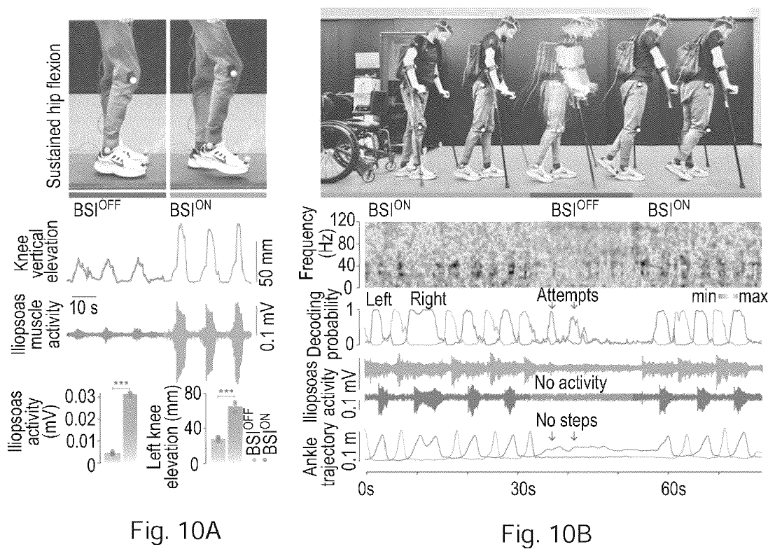

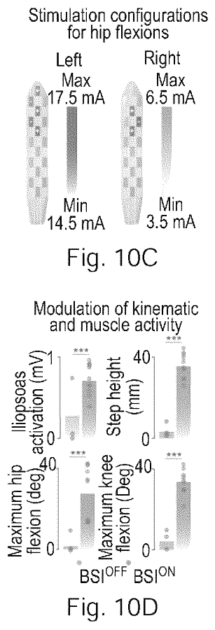

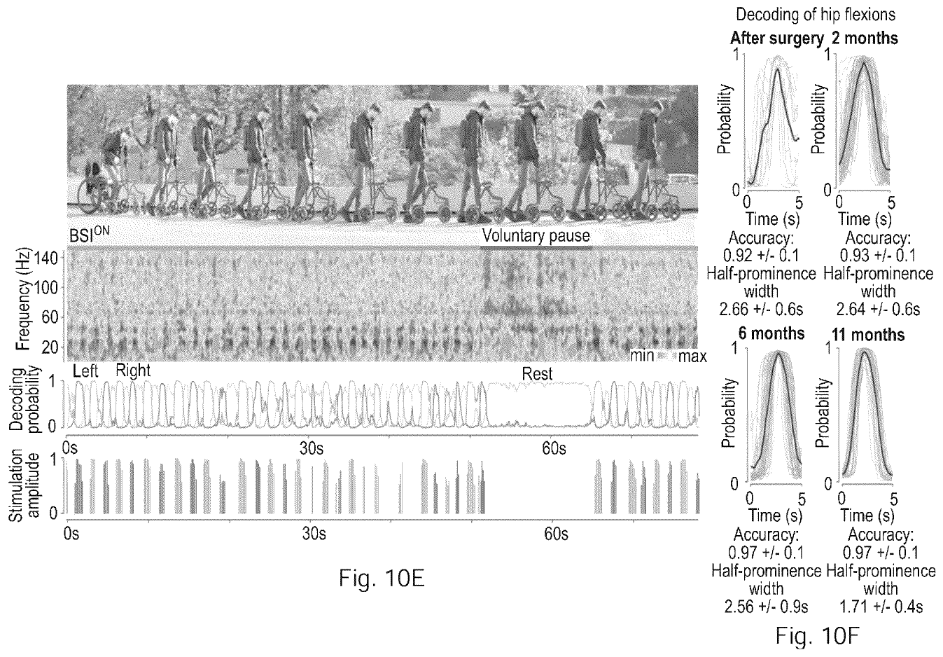

After only 5 minutes of calibration, the BSI supported continuous and intuitive control over the activity of hip flexor muscles, which allowed the participant to achieve a fivefold increase in muscle activity compared to attempts without the BSI (FIG. 10a).

The same configuration was provided to support walking with crutches.

The BSI enabled continuous, intuitive and robust control of walking (FIG. 10b).

When the BSI was turned off, the participant instantly lost the ability to perform any step despite detected attempts to walk from the modulation of cortical activity.

Walking resumed as soon as the BSI was turned back on.

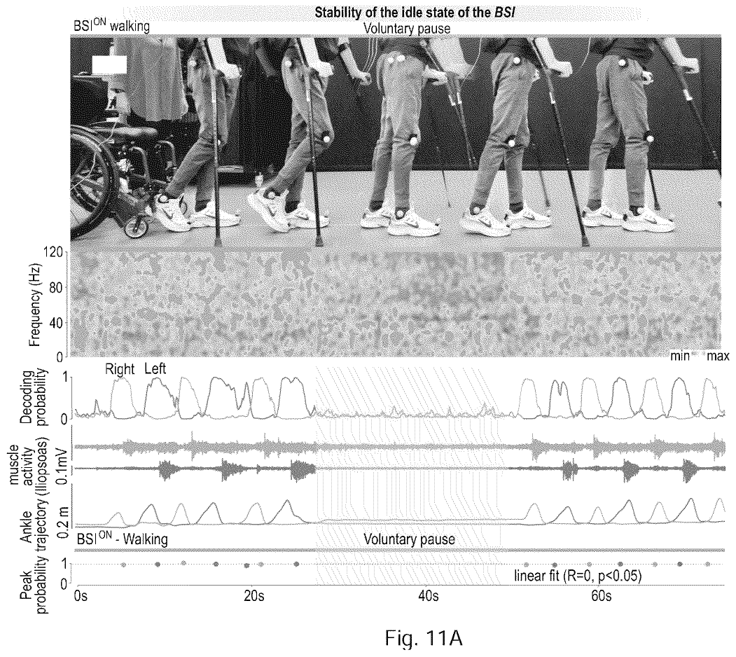

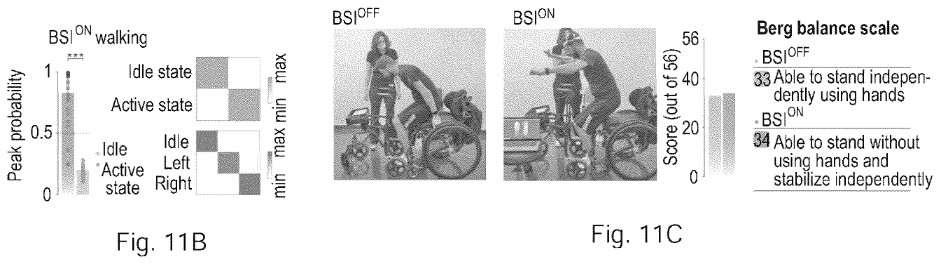

The participant was able to decide whether to initiate stepping, walk continuously, stop, or stand quietly without the detection of false positives that would impair standing performance (FIG. 11).

Indeed, Berg Balance Scale assessments revealed that the BSI did not impair, and even slightly improved overall balance abilities (FIG. 11c).

The participant reported that the BSI enabled natural control over his movements during walking.

One goal was to capture this subjective perception with quantified outcomes.

For this purpose, a principal component analysis was applied to whole body kinematics and muscle activity collected during walking on a treadmill with the BSI or with the same stimulation programs controlled in a closed-loop based on motion sensors attached to the feet.

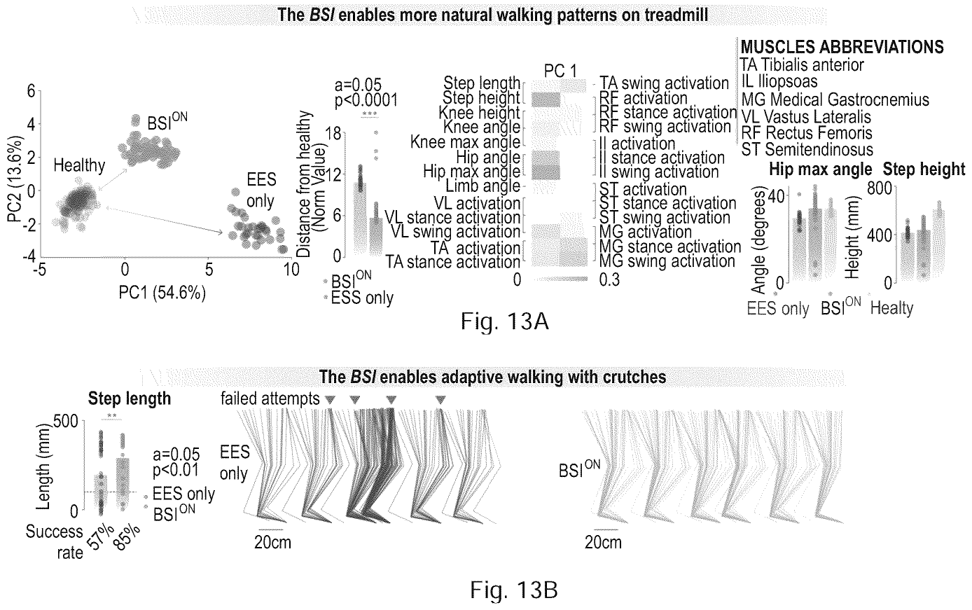

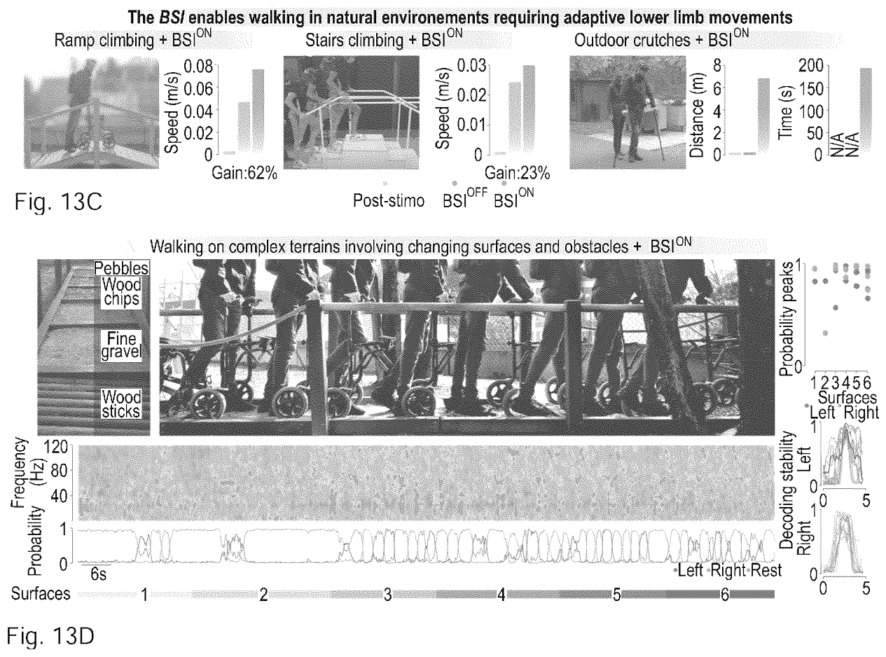

Compared to stimulation alone, the BSI enabled walking with gait features that were markedly closer to those quantified in healthy individuals (FIG. 13a).

The BSI ensured a continuous link between the intended movement and the modulation of stimulation protocols, which translated into the ability to walk overground independently with crutches.

When the intended movements were detected from the motion sensors, the participant reported a frequent temporal mismatch between the detections and his intentions, which impaired his ability to walk under these conditions (FIG. 13b).

### The BSI Enables Natural Walking on Complex Terrains

We next aimed to demonstrate that the BSI enabled intuitive and natural control over complex activities of daily living that were not possible without the BSI.

When the participant enrolled in the previous clinical trial, 7 years after his accident, he was not able to walk independently.

Completion of this clinical trial allowed him to regain basic walking when stimulation was turned on, albeit this recovery required compensatory strategies to trigger the sequences of stimulation based on heel elevations.

He also recovered partial mobility without stimulation.

However, he experienced difficulties transitioning from standing to walking and stopping, and could only walk over flat surfaces.

Moreover, he was not able to adjust lower limb movements to progress over ramps, overcome obstacles or climb up staircases—as necessary to support mobility in everyday life.

To demonstrate that the BSI remedied these limitations, a succession of paradigms was designed, that emulated the conditions underlying these activities of daily living.

With the BSI, the participant climbed up and down a steep ramp with ease, performing this task twice faster than without stimulation.

The BSI also enabled high step clearance, as necessary to climb over a succession of stairs, negotiate obstacles, and traverse changing terrains (FIG. 13c-d).

All these tasks were performed with the same BSI configuration, which proved highly reliable to support a broad variety of tasks with widely different constraints (FIG. 13c-d).

### Long-Term Stability of the BSI

The next aim was to assess the stability of the BSI.

For this purpose, the stability of cortical signals and decoders over time and the need to adjust stimulation programs were quantified.

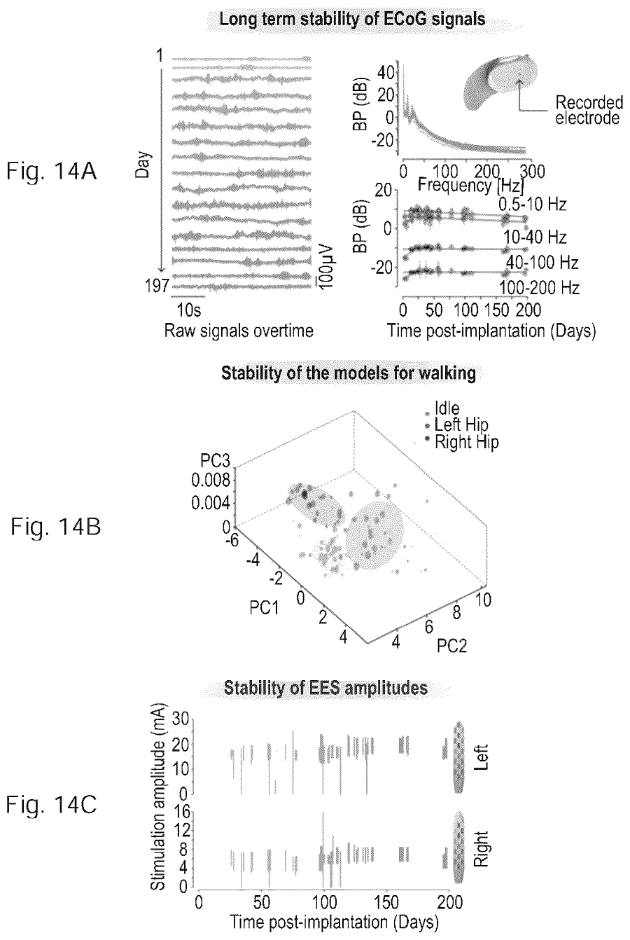

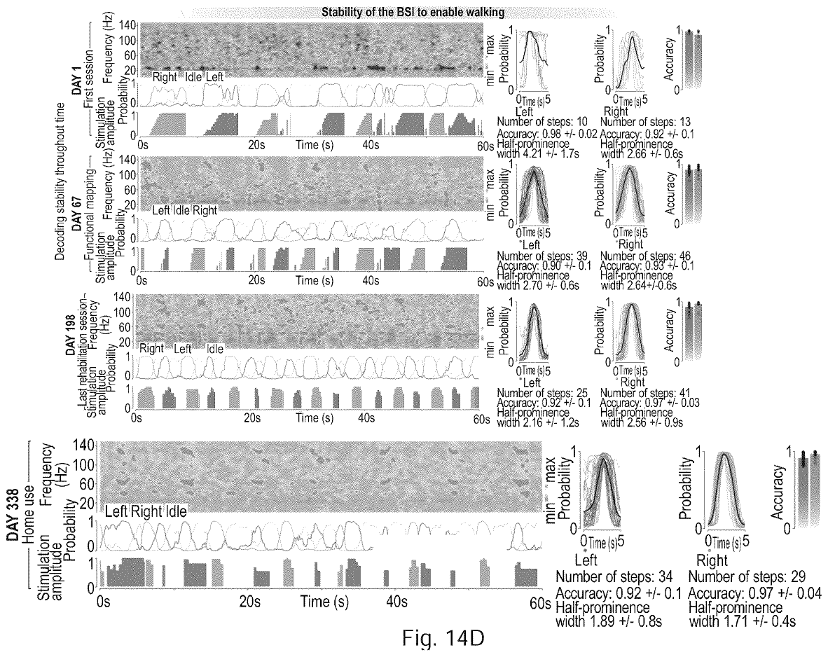

After a transitory 1-month period during which cortical signals exhibited modest changes in the spectral content of the different frequency bands, ECOG signals remained stable over the following months (FIG. 14a).

The decrease in the spectral power was limited to 0.03 dB/day on average.

This stability enabled robust performance.

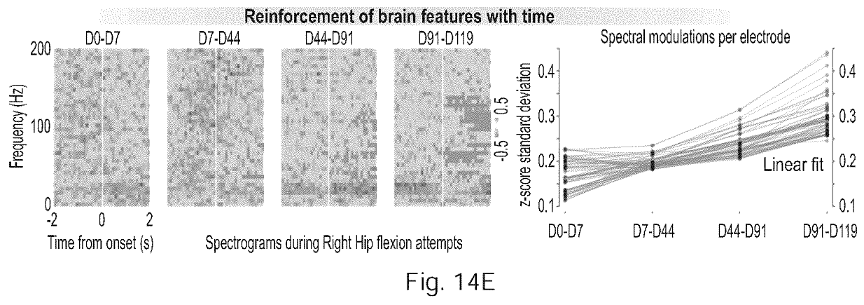

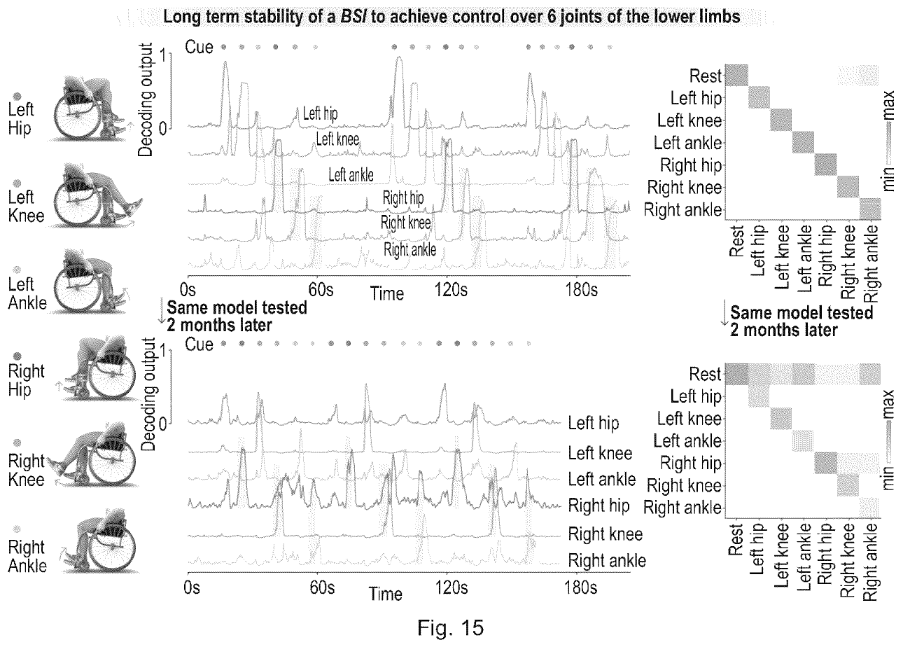

For example, it was found that the same decoder enabled the participants to achieve gradual control over 6 joints despite a 2-month interval between both sessions (FIG. 15).

This robustness was leveraged during neurorehabilitation since the BSI was only recalibrated when deemed necessary by the participant and/or physiotherapists in order to promote the best possible functional performance.

Despite these recalibrations, the features of the decoders remained remarkably stable over time (FIG. 14b).

Indeed, signal quality and decoding accuracy during walking has remained globally unchanged over nearly one year of use (FIGS. 10f, 14d).

While cortical features remained remarkably stable over time, we detected a progressive
reinforcement of their modulation depth that revealed gradual improvements in the ability of the participant to modulate his cortical activity (FIG. 14e).

The library of stimulation programs showed the same stability.

The optimal range of stimulation amplitudes was dependent on the specific configuration of electrodes and targeted muscles (FIG. 14c).

However, these ranges of stimulation amplitudes have remained stable over one year of use, and stimulation thresholds did not change over time.

### Neurological Recovery

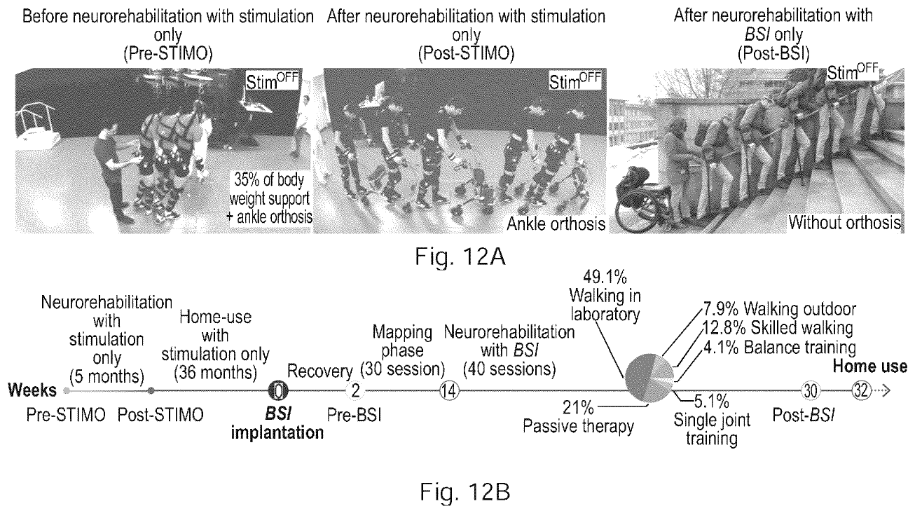

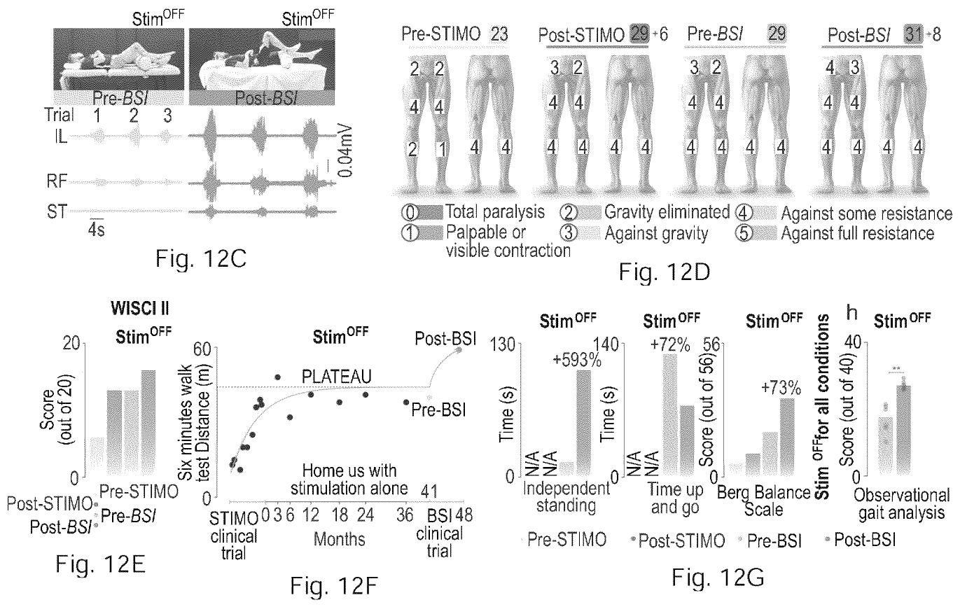

The clinical study was designed to investigate whether neurorehabilitation supported by the BSI further improves neurological recovery (FIG. 12a).

Before enrolling in the present clinical trial, the participant had completed the previous clinical trial, which allowed him to regain volitional control over previously paralyzed muscles and improve his standing and walking functions.

However, after three years of regular training with stimulation only, he had reached a plateau of recovery (FIG. 12d-f).

The participant completed 40 sessions of neurorehabilitation (FIG. 12b) that involved walking with BSI, single-joint movements with BSI, balance with BSI, and standard physiotherapy.

Since impairments were more pronounced in hip flexor muscles, the training exercises and BSI configurations were primarily focused on the control of these muscles.

This neurorehabilitation program mediated pronounced improvement in the volitional control of hip flexor muscles and associated hip flexion movements without stimulation (FIG. 12c).

This recovery correlated with gains in sensory (4 points in light touch sensory score) and motor scores (FIG. 12d), and enhanced standing and walking capacities that were captured in an increase in WISCI II scores from 6 prior to STIMO to 16 after STIMO-BSI (FIG. 12e).

Concretely, the participant exhibited improvements in all the conventional clinical assessments such as the 6-minute walk test, weight-bearing capacities, timed up and go, Berg Balance Scale, and walking quality assessed using the observational gait analysis scale 25 by physiotherapists blind to the study (FIG. 12d-h, Supplementary table 3).

These improvements translated into a meaningful increase in quality of life, such as walking independently around his house, transiting in and out of his car, or drinking a beverage with friends standing at a bar.

### Integration of the BSI in Daily Life

The BSI enhanced the standing and walking capacities of the participant, which led to the development of a BSI framework for independent use at home.

A system was designed that could be operated by the participant without any assistance.

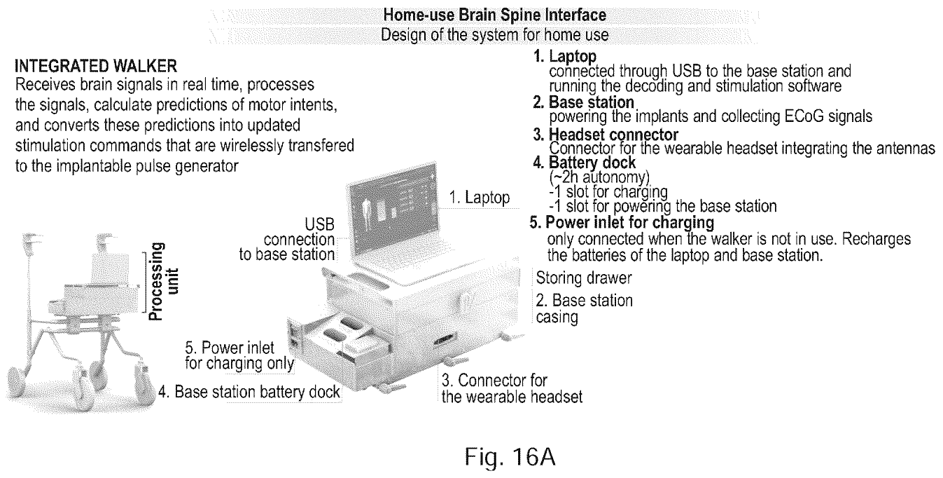

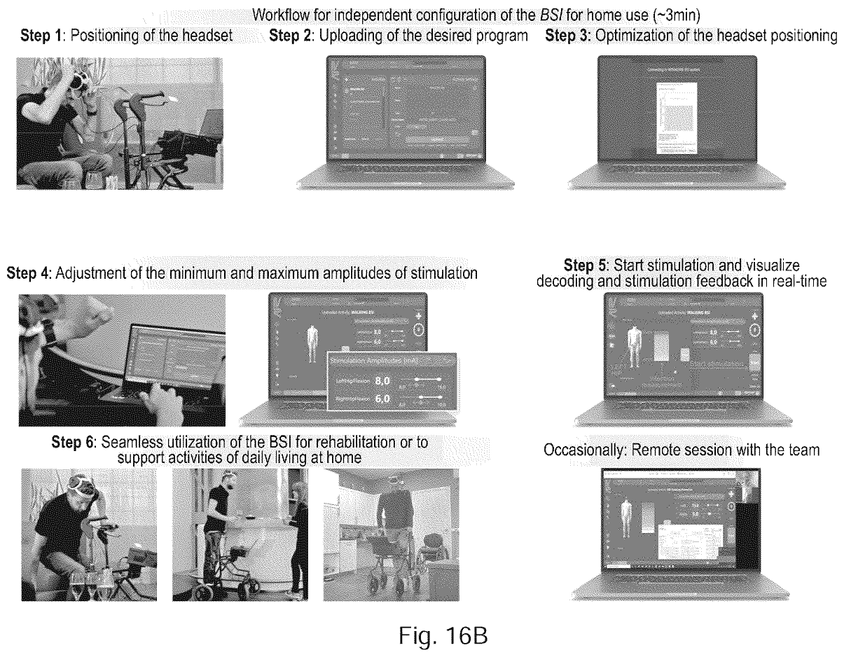

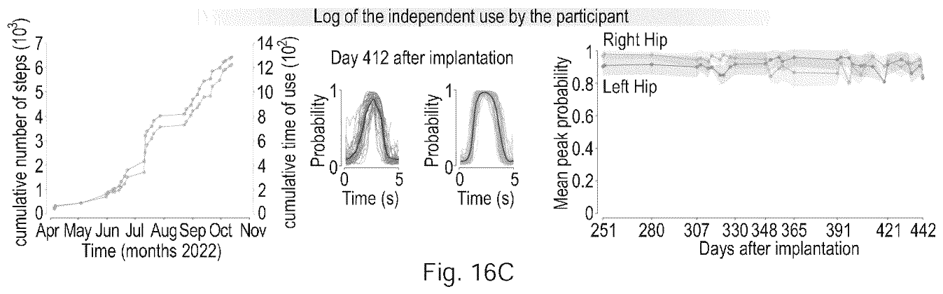

This system includes a walker equipped with an integrated case that embeds all the components of the BSI (FIG. 16).

A tactile-based interface allows the participant to interact with the tailored software in order to launch an activity, verify the placement of the headset, and adjust the minimum and maximum amplitudes of stimulation programs.

The configuration of the hardware and software was completed with minimal user inputs within less than 5 minutes, after which the participant can leverage the BSI for neurorehabilitation or to support activities of daily living.

The participant used the system regularly over the course of 7 months with stable decoding performance (FIG. 16c).

This home-use translated into a broad increase in the perceived benefits by the participants, as quantified by the Psychosocial Impact of Assistive Devices Scale (PIADS) questionnaire (Supplementary table 4).

Security, skillfulness and the ability to participate were ranked with the maximum possible gains in this questionnaire.

### Methods

### Study Design and Participant

All experiments were carried out as part of the ongoing clinical feasibility study investigating the safety and preliminary efficacy of brain-controlled spinal cord stimulation after SCI.

The study was approved by the Swiss authorities (Swissethics protocol number CER-VD2020-01814, Swissmedic 10000766, EUDAMED CIV-20-07-034126), and was carried out in accordance with the Declaration of Helsinki.

A written informed consent has been signed by the participant prior to participation.

All surgical and experimental procedures were performed at the Lausanne University Hospital (CHUV), with the sole exception of the magneto-encephalography experiments, that were carried out at the CEA Clinatec facilities (Grenoble, France).

The study involved functional assessments before implantation of the cortical devices, the neurosurgical procedure, a 6-week period during which various decoders were calibrated and spinal cord stimulation libraries were established, and a 15-week period of neurorehabilitation with physiotherapists that amounted to a total of 40 sessions, each lasting one to three hours.

The neurorehabilitation program was personalized based on the participant's improvements.

At the end of the neurorehabilitation period, the participant exited the active participation phase of the clinical trial and was offered the opportunity to continue using the system at home.

The participant is currently followed up on a regular basis by the study team until the end of a 3-year study extension phase of home use of the system.

As mentioned, prior to enrollment in the present clinical trial, the participant already participated in a previous clinical trial involving a 5-month neurorehabilitation program supported by targeted epidural electrical stimulation of the spinal cord, followed by a 2-year period of independent use at home.

Additionally, one year before joining the present clinical trial, the participant underwent a surgical procedure with:
(1) talonavicular arthrodesis, transfer of the toe-extensors to the m. peroneus tertius;
   and transfer of the m. tibialis posterior to the m. tibialis anterior and m. extensor digitorum longus, and
(2) tenotomy of all long toe-flexors and interphalangeal arthrodesis of the hallux.

Both surgical procedures were performed bilaterally and could have impacted the reliability of the long-toe extensor motor scores before and during the study due to changes in spasticity and mechanical properties of the joint.

Therefore, it has been decided not to report the LTE motor score in the analysis.

### Pre-Operative Magneto-Encephalography (MEG).

Before entering the clinical trial, the participant was already implanted with a spinal cord system that however was not MRI compatible.

Therefore, it was not possible to perform an anatomical or functional MRI of the brain.

MEG is less sensitive to anatomical imprecisions for source reconstruction compared to EEG [42].

Therefore, it was established to use MEG to map the activity correlated to limb motor intentions.

Prior to the neurosurgical procedure to place the cortical implants, MEG activity was measured in a magnetically shielded room using a 306-channel whole scalp array (204 planar gradiometers and 102 magnetometers) from the Elekta Neuromag system (Elekta Neuromag Oy, Helsinki, Finland).

ECG and EOG were recorded simultaneously.

The recording sampling rate was 1000 Hz.

Continuous head position indicator (CHPI) signals were recorded during the experiments to track the subject's head movements.

Prior to experimentation, a 3D digitization system (Isotrak II®, Polhemus, Colchester, VT, USA) was used to localize anatomical fiducial points for later co-registration with head computerized tomography (CT).

Temporal signal space separation (tSSS) was applied to reduce the noise in the MEG data using MaxFilter 3.0 software (Elekta, Helsinki, Finland).

First, a manual review of raw data allowed the marking of bad channels.

Second, the tSSS filter was applied using head movement compensation and automatic bad channel correction.

The main parameters were kept to default (tSSS correlation threshold of 0.98, orders of expansion for “in” and “out” components of signal respectively set to 8 and 3, and a 10 s-long-time buffer).

Notch filtering at 50 Hz and harmonics (100 Hz, 150 Hz, 200 Hz and 250 Hz) was also applied to remove power line contamination.

Stereotypical artifacts (cardiac, ocular) were identified by independent components analysis using MNE-Python software [43] and rejected upon visual screening (Infomax method, calculated separately for magnetometers and gradiometers using 64 components).

The head, skull and cortex geometries were calculated from CT scan using an MRI segmentation routine included in Brainstorm software [44], followed by the calculation of the head model using the overlapping spheres method.

A 3D inversion kernel was calculated using Brainstorm implementation of Minimum Norm Imaging method with default parameters.

This allowed the reconstruction of cleaned raw data at the brain source level for subsequent calculations.

The MSA method was finally used to reconstruct the brain activity related to wrist, hip and ankle motor attempts.

To estimate task-specific brain activations, MSA uses cross-validated, shifted, multiple Pearson correlation, calculated from the time-frequency transformed brain signal and the binary signal of stimuli.

3D snapshots of these activations at their maximum were exported as DICOM in the original CT-scan frame of reference for use in neuro-navigation tools. For rendering, we segmented manually the brain from the patient's pre-operative CT-scan using Slicer (slicer.org) and used Blender for rendering.

The MEG signal was recolored with a red color ramp and then superimposed with the 3D render of the brain.

### Electrocorticography Devices.

A WIMAGINE® implantable recording system was used to carry out the present trial.

The WIMAGINE® implantable recording system was designed for bilateral epidural implantation over the sensorimotor cortex [16], [52]-[55].

Electronic components are housed in a titanium case (50 mm diameter, 7-12 mm thick, and a convex external face).

An array of 64 platinum-iridium (90:10) recording electrodes for epidural ECOG (2 mm in diameter, 4 and 4.5 mm pitch) and five reference electrodes are located on the flat inner face of the device.

The ECOG data were recorded thanks to an application-specific integrated circuit (ASIC) enabling multi-channel amplification and digitalization with an input referred noise of less than 0.7 μVRMS in the [0.5 Hz-300 Hz] range.

Data were radio-emitted through an ultra-high frequency antenna (402-405 MHz).

Power was supplied remotely through a 13.56 MHz inductive high-frequency antenna.

Both antennas were embedded in a silicone flap extending on the subcutaneous space.

To ensure signal stability at a high frequency (586 Hz), in regard to the limited bandwidth, 32 contacts out of the 64 were used for each implant.

The wireless connection used two external antennas held in front of the recorders by a custom-designed headset.

The technical specifications of the device are reported in Supplementary table 1.

### Neurosurgical Procedure

Surgery was performed under general anaesthesia.

A neuro-navigation station (StealthStation, Medtronic, Minneapolis) was used to locate the center of the craniotomies on each hemisphere.

The anatomical and functional information obtained from MEG and CT-scan imaging allowed the selection of the entry points in order to maximize the coverage of the leg region of the sensorimotor cortex while ensuring the same margin from the sagittal sinus area.

Following a coronal incision, two circular craniotomies of 5 cm in diameter were performed using a custom-made trephine.

The bone flaps were removed to expose the dura matter.

The two WIMAGINE® implants were placed over the dura, and then carefully suspended and secured with non-resorbable sutures.

The skin was then sutured over the implants.

The participant was discharged the next day.

The calibration phase was initiated after a rest period of 2 weeks.

### Decoder Architecture

The used decoder architecture is of the kind described, e.g., in U.S. Pat. No. 10,832,122A1 and EP3789852A1.

ECOG data were collected from 32 channels per implant at the acquisition frequency of 586 Hz.

The signals were bandpass-filtered between 0.5 Hz and 300 Hz.

The data was streamed through the field trip toolbox to a custom-made decoding software running in Matlab Runtime Environment (The Mathworks Inc).

To decode the intention to perform lower-limb movements, a variant of the recursive exponentially weighted Markov-switching multi-linear model (REW-MSLM) algorithm previously developed to decode upper-limb movements [23] was implemented.

REW-MSLM is a mixture of multilinear experts' algorithms.

It consists of a Hidden Markov Model (HMM) classifier, called a gate, and a set of independent regression models called experts.

Each expert is generally dedicated to the control of a group of degrees of freedom, a specific limb or movement (e.g. joint movement).

The HMM-based classifier predicts the probability of such specific limb or movement activation (states) associated with a particular expert.

The resulting decoder output issues from soft mixing of expert predictions according to estimated probabilities.

The gate HMM-based classifier predicts the state and assumes that the state sequence Z(t) follows a 1^{st}-order Markov chain hypothesis.

Consequently, the probability of a state at each time step depends on the combination of the previous state and the newly acquired ECoG data.

A HMM-based classifier is composed of an emission and transition probability model.

At each time step, the emission probability is estimated from the observations of ECOG signals independently from the sequence of the state.

In the current study, a linear discriminative classifier is employed for the emission probability model.

For K states/classes (in the present case, K=7 states, rest, hip, knee, ankle, bilaterally), the classifier output is computed as follows:

$${d}^{gate} ( t ) = {β}^{gate} X ( t ) + {b}^{gate} ,$$

$${d}^{gate} ( t ) = ( {d}_{1}^{emission} ( t ) , … , {d}_{K}^{emission} ( t ) )$$

Here, β^{gate} and b^{gate} are matrices of coefficients of linear discriminative classifiers.

Then, the emission probability vector

$${α}^{emission} ( t ) = ( {α}_{1}^{emission} ( t ) , … , {α}_{K}^{emission} ( t ) )$$

is obtained from the classifier output d^{gate}(t) the softmax normalization:

$${α}_{k}^{emission} ( t ) = \frac{{e}^{{d}_{k}^{gate} ( t )}}{{\sum }_{i = 1}^{K} {e}^{{d}_{i}^{gate} ( t )}} .$$

Finally, the emission probabilities are weighted by the HMM state transition probabilities matrix T, where T is a K-by-K matrix with coefficients defined by the cumulative number of transitions between cued states.

$$\hat{α} ( t ) = \frac{{α}^{emission} ( t ) \times T \cdot \hat{α} ( t - 1 )}{‖ {α}^{emission} ( t ) \times T \cdot \hat{α} ( t - 1 ) ‖} .$$

The most probable state \hat{Z}(t) sequence may be issued by maximizing the state probability \hat{α}(t) at step t.

The state probability vector may be used to mix the decoder experts or may be considered as one of the decoder outputs.

For experts, a multilinear regression model is employed:

$${φ}_{k} ( t ) = {β}_{k}^{expert} X ( t ) + {b}_{k}^{expert}$$

where

$${β}_{k}^{expert} and {b}_{k}^{expert}$$

coefficients of k^{th} expert, k∈[1, K]. Finally, the mixture of expert output U(t) is computed from expert predictions φₖ(t) and estimated probabilities \hat{α}ₖ(t) from the following equation: Uₖ(t)=φₖ(t)×\hat{α}ₖ(t)×Π_{i≠k}(1−\hat{α}ᵢ(t)).

From this decoder architecture, two different control paradigms were implemented to drive epidural spinal cord stimulation.

In particular:

i) For six-joint control in static conditions, a mixture of expert predictions was implemented to enable the participant to achieve proportional control over the amplitude of stimulation. U (t) contains the analog prediction of the relative desired amplitude of joint movement at any given time. The movement of each joint is linked to a specific stimulation protocol (electrode configuration, frequency and pulse width) defined in the library of stimulation programs provided in FIG. 9b-d, while the predictions constituting U (t) are linearly rescaled into the amplitude of stimulation (in mA) within a range of predefined values by the experimenter.

ii) For the control of standing and walking in dynamic conditions, it has been considered that these activities do not require simultaneous control over the amplitude of left and right hip flexions, since left and right steps must not occur at the same time.

Therefore, the layer of a mixture of experts was removed, and a control paradigm that drives stimulation amplitudes from the gate model output has been implemented instead.

This control paradigm avoids the simultaneous delivery of stimulation over all the joints.

Consequently, the amplitude is only modified for one joint at a time.

In turn, the maximum estimated state probability max (@ (t)) was used to enable the participant to achieve proportional control over the amplitude of stimulation.

### Iterative Online Decoder Calibration.

REW-MSLM is a closed-loop adaptive decoder.

In parallel to the current model used for predictions, the REW-MSLM decoders update their parameters based on new incoming data, which allows the optimization of the parameters of the model in real-time throughout the calibration session [23].

The linear emission probability model and expert models were identified using a recursive exponentially weighted N-way partial least square (REWNPLS) algorithm.

This algorithm was specifically designed for incremental and adaptive real-time multilinear decoder learning [47].

The transition matrix is identified by direct state transition counting during the calibration session. The resulting decoder is able to predict mental states as well as continuous movements.

Input features X(t) are computed from the ECOG signals and then fed to the decoder. Epochs ranging from 200 to 500 ms of ECOG signals from the 64 electrodes were created to generate a 100 ms sliding window.

The epochs are mapped to the temporal-frequency space with complex continuous wavelet transform (CCWT).

The wavelets declined from the Morlet mother wavelet are centered around specific frequencies [2 5:5:100 125 150 200] Hz.

The absolute value of the output of the CCWT is then decimated to obtain 2-5 points along the temporal modality, which defined the epoch.

The prediction is computed every 100 ms.

During the experiments, the REW-MSLM algorithm recursively updates the experts and gate coefficients every 15 s.

The training data consists of 15 s batches of input ECoG features associated with output movement features.

The output features are generated according to specific tasks given to the patient to perform motor imagery including desired mental state for the gate update and a desired continuous movement (if any) for the expert in charge update.

When creating a model from scratch, assistance from the system can be added to the decoder output.

This allows the patient to have movements already performed, even though the decoder does not predict correctly.

Assistance decreases progressively as the model is calibrated to eventually leave the patient in full control.

### Model Update:

Step 1: Accumulate raw data and label d^{gate} and φ over 15 s.
Step 2: Compute the corresponding feature vector X(t)
Step 3: Perform the partial least square regressions for the gating
d^{gate} and experts q to update the coefficients (β^{gate}, b^{gate}, β^{expert}, b^{expert}).
Step 4: Update the transition matrix by adding the number of transitions T(i,j)<−T(i,j)+sum((Z(t+1),Z(t))=(i,j))

### Prediction Computation:

Step 1: Compute the linear predictions of gate and experts from the current coefficients.
Step 2: Apply the exponential normalization and the HMM transition step.
Step 3: Mix the prediction from the gating and expert model.

### ECOG Mapping Procedure

To assess the spatial and spectral information in the ECOG signals to discriminate a specific task the linear regression weights were computed associated with cued motor attempts.

To map the features related to lower limb movements (hip, knee, ankle bilaterally) we recorded cortical signals during 57 (+−6, s.e.m.) repetitions of each movement attempt cumulating 226 s (+−25 s, s.e.m.) during each state.

The weights generated from this dataset were projected onto the spatial dimension for different frequency bands (0.5-10 Hz, 10-40 Hz, 40-100 Hz and 100 to 200 Hz) or on the spectral dimension.

### Epidural Electrical Stimulation

The implant to deliver epidural electrical stimulation (Supplementary table 2) was composed of an ACTIVA RC implantable pulse generator (IPG) (Model 37612, Medtronic, USA) that is interfaced with the Specify Surescan 5-6-5 paddle lead (Model 977C190, Medtronic, USA).

A dedicated firmware enables real-time uploads of stimulation tables to control electrical stimulation waveforms [8].

The patient programmer (SPTM, Model 09103, Medtronic, USA) was carried within a belt in order to align its position with the IPG.

A custom-built stimulation program [8] was built, that sent commands to the patient programmer through a Bluetooth/Infrared wireless bridge.

The stimulation program enabled the definition of stimulation configurations (cathodes and anodes) and parameters (pulse width, frequencies and amplitude ranges) by an expert user [8].

This chain of software and hardware enabled real-time control of stimulation protocols with a latency inferior to 150 ms [7].

### Calibration of the Library of Stimulation Programs

Electromyographic (EMG) activity was recorded bilaterally from the iliopsoas (II), rectus femoris (RF), vastus lateralis (VLat), semitendinosus (ST), tibialis anterior (TA), medial gastrocnemius (MG), and soleus (Sol) muscles with wireless bipolar surface electrodes (Delsys Trigno, UK).

Each pair of electrodes was placed over the belly of the targeted muscle and aligned longitudinally to muscle fibers.

An abrasive paste (Nuprep, 4Weaver and Company, Aurora, CO) was used for skin preparation to reduce electrode-skin resistance and improve EMG signal quality.

An additional pair of surface-EMG electrodes were placed over the spine, at the thoracolumbar junction, to detect stimulation artefacts, and thus align muscle responses to the onset of stimulation.

Continuous EMG signals were sampled at 2 kHz and saved to a desktop computer.

EMG signals were band-pass filtered between 20 and 450 Hz.

Recruitment curves were performed with pulses of increasing stimulation amplitudes, delivered every second.

A grid search paradigm was implemented to explore the different electrode configurations and frequencies in order to select the configurations of cathodes and anodes to achieve maximum selectivity in the recruitment of the targeted muscle groups [7].

The amplitude of muscle responses was normalized by z-scoring over all the configurations.

For each period of stimulation, the average absolute value of the z-score was computed.

The z-score was then represented in a polar plot.

### Decoder Calibration Procedure

During the calibration of the decoders, the participant received visual cues through a custom-made interface displaying the targeted state with or without the direction of movement.

The cues were generated as a pseudo-random sequence with programmable duration (2/4 s per state) or manually by the experimenter.

The decoding environment enabled visualization of the duration spent in each state as well as the number of transitions between states.

Once the performance of the decoder was judged sufficient by the participant and experimenter, cueing was discontinued and the participant could use the model without further calibration.

From day to day, models were updated when deemed necessary.

The iterative nature of the implementation facilitated these updates.

Typically, the model supporting control over left hip and right hip flexions during walking was trained based on 30 repetitions of each active state, while the resting state was predicted from 3 minutes of ECoG data that were acquired while the participant was performing unspecific hand and trunk movements as well as talking in order to ensure the robustness of the predictions.

### Decoding Accuracy Quantification

The accuracy of decoding predictions was quantified by computing the normalized cross correlation between the decoded state \hat{Z} and the cued state Z after delay compensation:

$$decoding accuracy = 1 / {\sum }_{t} Z ( t ) \cdot {\sum }_{t} Z ( t - τ ) \cdot \hat{Z} ( t )$$

where τ corresponds to the time at which the maximum of the cross-correlation between the cued state and the decoded state probability is reached.

### Muscle Response Accuracy Quantification

Accuracy of muscle responses was obtained by computing the normalized cross correlation between the decoded state \hat{Z} and the thresholded EMG envelope, which was obtained with T=200 ms sliding window:

$$EMG ( t ) = [ \frac{1}{T} {\int }_{t}^{t + T} \vert zscore ( emg ( τ ) ) \vert d τ ] > 1$$

$$emg accuracy = 1 / {\sum }_{t} EMG ( t ) \cdot {\sum }_{t} EMG ( t ) \cdot \hat{Z} ( t )$$

### Stepping Accuracy Quantification

When walking freely, there was no cue to quantify the decoding accuracy.

To provide a quantification, the probability curves that were decoded during walking were profiled, and the peak value and width of the probability curves associated with the left and right hip flexions were analyzed.

This analysis was conducted during walking at different time points, from the first session to sessions that occurred nearly one year after the neurosurgical procedure to place the cortical implant.

The mean peak of probabilities as well as the mean half-width (+/−standard deviations) were calculated for each time point.

### ECoG Spectrograms

To generate spectrograms of ECOG signals, a continuous wavelet transform with a window of 500 ms and a step size of 100 ms was applied.

The difference between the averaged spectrograms from both implants was computed and normalized by applying a z-score over each frequency band.

The colormap of the normalized average spectrograms were scaled between −0.5 and 0.5 or between −0.5 and 1 for visualization.

### ECoG Signal Stability

Signal stability was assessed by quantification of the signal power during the resting state in the different frequency bands [48].

The participant was sitting in his wheelchair with his eyes closed while ECOG signals were acquired for 2 minutes.

For each session, a window of 90 s starting 20 s after the onset of recordings was selected for analysis.

The power spectrum density (PSD) was estimated using Welch's method.

Root Mean Square (RMS) was computed over the entire frequency band (0.5-292 Hz).

The band power was measured for the following four frequency bands: 0.5-10 Hz, 10-40 Hz, 40-100 Hz and 100-200 Hz.

To compensate for the different frequency bandwidths, the obtained band powers were normalized before being converted into dB.

The signal-to-noise ratio (SNR_{band}) was calculated for each band as the ratio of the band power versus the noise band power that was estimated between 250-260 Hz due to the numerical filter.

RMS, BP and SNR_{band} were finally averaged across all the electrodes.

Feature Reinforcement with Time

The reinforcement of cortical features linked to hip flexion attempts was analyzed by computing the median spectrograms around the flexion cues in 4 different time periods to gather 100 events per period (−2 s to +2 s around the event).

For each electrode, we computed the standard deviation of the spectrograms over all frequencies during the 4 s surrounding the events.

A linear fit was performed over the 64 electrodes and 4 time periods.

### Walking Model Stability Assessment

To analyze the stability of the walking models, a principal component analysis (PCA) was applied over the coefficients of each gate (Idle, Left Hip Flexion, Right Hip Flexion) for each model.

The gate vectors were composed of (64 channels×24 frequencies) coefficients by averaging the temporal dimension.

The PCA was performed over (3 gate×44 models) samples spanning 4 months of use.

Data were represented in the first 3 components of the PCA.

An ellipsoid of 1′600 data points was constructed, representing the contour curve that corresponded to a standard deviation of 1.4 for a 3-dimensional Gaussian distribution with the covariance and the mean value of each state.

### Quantitative Gait Analysis

EMG activity during walking was acquired bilaterally at 1259 Hz using 16-channel wireless sensors (Delsys Trigno, UK) placed over the iliopsoas (II), rectus femoris (RF), vastus lateralis (VLat), semitendinosus (ST), tibialis anterior (TA), medial gastrocnemius (MG).

Kinematic recordings were acquired using a 3D motion capture system (Vicon Motion Systems, Oxford, UK).

A network of 14 infrared cameras, which covered a 12×4×2.5 m workspace, was used to record the motion of markers attached to body landmarks.

Data were acquired at a 100-Hz sampling rate using.

A principal component analysis was applied over a total of 26n kinematic and EMG parameters that were calculated for each gait cycle, as described previously [7].

The following parameters were included: step length, step height, knee height, knee angle and knee maximum angle, hip angle and hip maximum angle, limb angle, VLat activation, VLat stance activation, VLat swing activation, TA activation, TA stance activation, TA swing activation, RF activation, RF stance activation, RF swing activation, Il activation, Il stance activation, Il swing activation, ST activation, ST stance activation, ST swing activation, MG activation, MG stance activation, MG swing activation.

Data were quantified during walking with the BSI and with closed control of stimulation based on motion sensors attached to the feet [8].

These data were compared to identical recordings obtained in healthy individuals.

During overground walking with crutches, stepping attempts were detected when the knee angle dropped below 135 degrees with at least 2 s between steps. Steps were considered as failed when the step length was lower than 10 cm.

### Observational Gait Analysis.

To analyze gait quality from video recordings, a panel of physiotherapists (n=6) who were blind to experimental conditions and were not involved in both the present and/or the previous clinical trials were asked to score different walking trials using items in a validated scoring sheet that is described in Supplementary table 3.

This scoring sheet pooled items from the validated questionnaires G.A.I.T. [25], SCI-FAI [49], Tinetti Test and reference [51].

### International Standards for Neurological Classification of Spinal Cord Injury (ISNCSCI)

Neurological status was assessed by an experienced neurologist based on the ISNCSCI, a comprehensive clinician-administered neurological examination of residual sensory and motor function quantifying SCI severity.

### Six-Minute Walk Test

Endurance was assessed by the distance covered overground within six minutes with a standard four-wheel walker but without any external assistance.

This test was performed before and at the end of each period of neurorehabilitation of the previous clinical trial and the present clinical trial.

Data were fitted with an exponential curve.

### 10-Meter Walk Test

Walking speed was assessed by a timed 10-meter walk test without any external assistance.

The participant was instructed to walk with the preferred assistive device as fast as he could.

### Statistical Analysis

Individual data points are represented in each figure.

Measurements were taken from distinct samples except for the observational gait analysis where the expert physiotherapists were independently ranking the same videos.

Paired or unpaired one-tailed t-tests were used, except otherwise specified, with alpha=0.05. P-values are reported with ***, p>0.001, **, p<0.01 and *, p<0.05.

### Device Explantation

Due to a subcutaneous infection of S. Aureus at the location of the cortical implant located on the right side, the Principal Investigator decided to explant the device 167 days after implantation.

The second implant presented no sign of infection and remained in place, fully functional.

After recovery from the surgery and antibiotics treatment per os, neurorehabilitation and daily use could continue as planned by the protocol.

Implantation of a new cortical implant is planned.

Note that the example control and estimation routines included herein can be used with various neuromodulation and/or neurostimulation system configurations. The control methods and routines disclosed herein may be stored as executable instructions in non-transitory memory and may be carried out by a control unit such as a microcontroller (or a computer) in combination with a processing unit 10, an implantable stimulation unit 12, an implantable pulse generator (IPG) 14, an implantable neurosensor 16, a first external antenna (high frequency, HF) 18, a second external antenna (ultra-high frequency, UHF) 20, a wearable device (head mounted device) 22 or a wearable device 24. The specific routines described herein may represent one or more of any number of processing strategies such as event-driven, interrupt-driven, multi-tasking, multi-threading, and the like. As such, various actions, operations, and/or functions illustrated may be performed in the sequence illustrated, in parallel, or in some cases omitted. Likewise, the order of processing is not necessarily required to achieve the features and advantages of the example embodiments described herein but is provided for ease of illustration and description. One or more of the illustrated actions, operations and/or functions may be repeatedly performed depending on the particular strategy being used. Further, the described actions, operations and/or functions may graphically represent code to be programmed into non-transitory memory of a computer readable storage medium in a control unit (e.g. a microcontroller) of the system, where the described actions are carried out by executing the instructions in a system including the various hardware components in combination with an electronic control unit.

### Neurosurgical Implantation of the BSI to Restore Upper Limb Movements.

Using the same approach described above (see method section for “lower limbs”), one participant with cervical spinal cord injury (46y.o. C4-AIS D) was implanted with a system according to the invention, combining the WIMAGINE® ECOG recording device and two implantable pulse generators (IPGs) connected to two epidural leads implanted over the cervical area.

In particular, the system was designed to restore voluntary control over up to five different movements including elbow extension, shoulder abduction, pronation, and hand opening/closing.

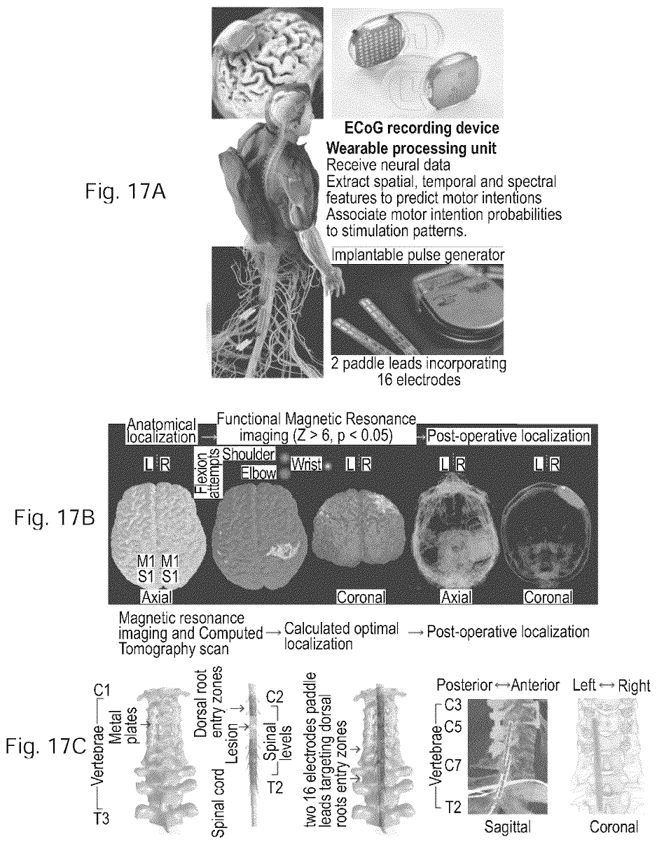

Implantation of the system is illustrated in detail in FIG. 17.

The clinical trial involved 50 neurorehabilitation sessions carried out using the system of the invention.

The clinical trial revealed that this neurorehabilitation training triggered significant neurological and functional recovery in the participant.

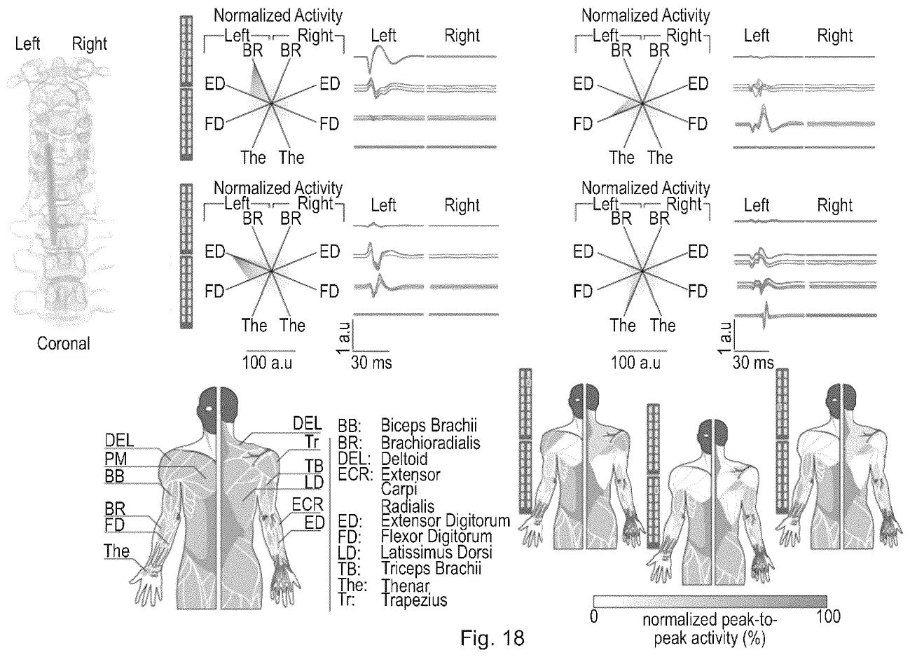

EES delivered to the cervical dorsal roots enabled selective upper limb muscle activation, as shown in FIG. 18.

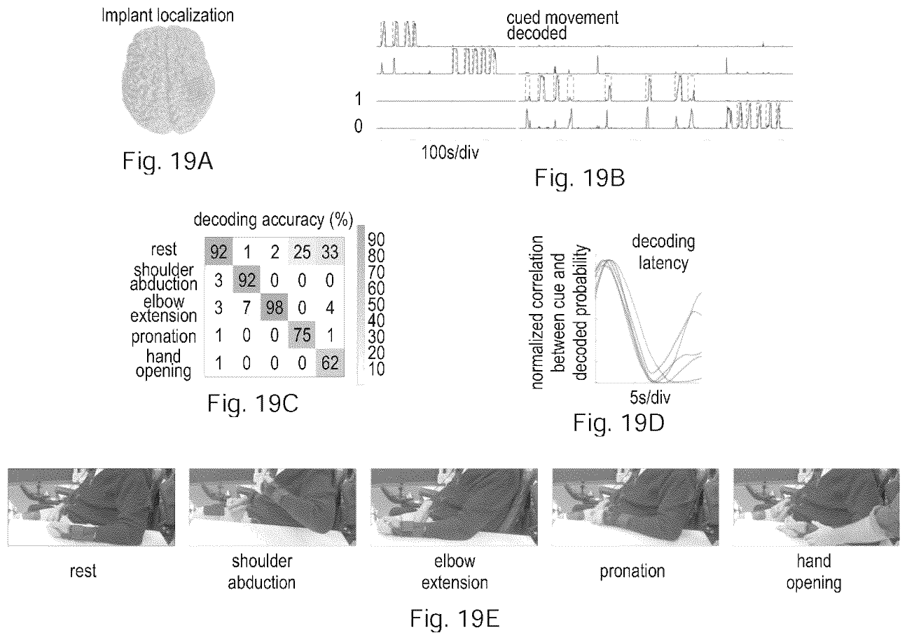

Decoding performance in the context of the present clinical trial is shown in detail FIGS. 19a-e.

FIG. 19a shows spatial localization of the cortical electrodes. Calibration of the system enabled discrimination between shoulder abduction, elbow extension, pronation, and hand opening, with an average 84% accuracy (+/−7%, s.e.m) and a latency of 1.3 s (+/−0.2 ms, s.e.m.) (FIGS. 19b-d).

In particular, latency (τ) was calculated as the peak time in the cross correlation between the cued movement and the decoding probability of this state.

The accuracy of decoding predictions was quantified by computing the normalized cross correlation between the decoded state \hat{Z} and the cued state Z after delay compensation:

In particular, decoding accuracy was defined as follows:

$$decoding accuracy = 1 / {\sum }_{t} Z ( t ) \cdot {\sum }_{t} Z ( t - T ) \cdot \hat{Z} ( t ) ,$$

where τ corresponds to the time at which the maximum of the cross-correlation between the cued state and the decoded state probability is reached.

FIG. 19e shows still shots illustrating the movements (namely: shoulder abduction, elbow extension, pronation, and hand opening) generated in the participant starting from a neutral position (rest).

The experimental evidence shown in FIG. 20 demonstrates that stimulation of the cervical region by using the system of the invention allows enabling reaching movements.

Here, decoding of elbow extension attempts are translated into epidural spinal cord stimulation triggering significant triceps activation (FIG. 20a, left side).

When the stimulation is turned off, although attempts are requested, reaching cannot be achieved.

FIG. 20b provides a comparison between muscle activity (maximum of rectified emg envelope) as well as joint angle with and without use of the BSI, demonstrating that use of the BSI allows achieving a significant increase of muscle activation and range of motion during movement attempts (p>0.001, unpaired t-test, n=5 repetitions).

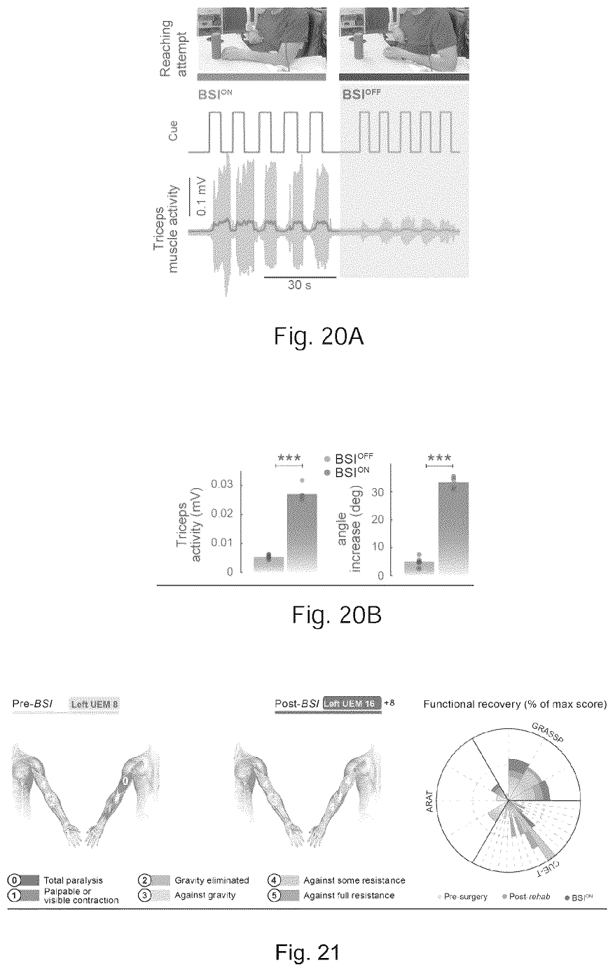

At the end of the 50 rehabilitation sessions, neurological and functional recovery were noted in the participant. In particular, the participant was able to gain 8 points over the motor score of the treated arm (FIG. 21).

### REFERENCE LIST

100 Neuromodulation/neurostimulation system
10 Processing unit
12 Stimulation unit
14 Pulse generator
16 Implantable neurosensor
18 First external antenna (high frequency, HF)
20 Second external antenna (ultra-high frequency, UHF)
22 Wearable device, head-mounted device (for the first and second external antennas)
24 Wearable device (for the processing unit)
P Patient
SC Spinal cord (patient)
C Sensorimotor cortex (patient)

### BIBLIOGRAPHY

[1] Arber, S. & Costa, R. M. Connecting neuronal circuits for movement. Science 360, 1403-1404 (2018).
[2] Courtine, G. & Sofroniew, M. V. Spinal cord repair: advances in biology and technology. Nat. Med. 25, 898-908 (2019).
[3] Capogrosso, M. et al. A computational model for epidural electrical stimulation of spinal sensorimotor circuits. J. Neurosci. Off. J. Soc. Neurosci. 33, 19326-19340 (2013).
[4] Wenger, N. et al. Closed-loop neuromodulation of spinal sensorimotor circuits controls refined locomotion after complete spinal cord injury. Sci. Transl. Med. 6, 255ra133 (2014).
[5] Wenger, N. et al. Spatiotemporal neuromodulation therapies engaging muscle synergies improve motor control after spinal cord injury. Nat. Med. 22, 138 (2016).
[6] Moraud, E. M. et al. Mechanisms Underlying the Neuromodulation of Spinal Circuits for Correcting Gait and Balance Deficits after Spinal Cord Injury. Neuron 89, 814-828 (2016).
[7] Wagner, F. B. et al. Targeted neurotechnology restores walking in humans with spinal cord injury. Nature 563, 65-71 (2018).
[8] Rowald, A. et al. Activity-dependent spinal cord neuromodulation rapidly restores trunk and leg motor functions after complete paralysis. Nat. Med. 28, 260-271 (2022).
[9] Capogrosso, M. et al. A brain-spine interface alleviating gait deficits after spinal cord injury in primates. Nature 539, 284 (2016).
[10] Minev, I. R. et al. Biomaterials. Electronic dura mater for long-term multimodal neural interfaces. Science 347, 159-163 (2015).
[11] Bonizzato, M. et al. Brain-controlled modulation of spinal circuits improves recovery from spinal cord injury. Nat. Commun. 9, 3015 (2018).
[12] Lorach, H., Charvet, G., Bloch, J. & Courtine, G. Brain-spine interfaces to reverse paralysis. Natl. Sci. Rev. 9, nwac009 (2022).
[13] Willett, F. R., Avansino, D. T., Hochberg, L. R., Henderson, J. M. & Shenoy, K. V. High performance brain-to-text communication via handwriting. Nature 593, 249-254 (2021).
[14] Vansteensel, M. J. et al. Fully Implanted Brain-Computer Interface in a Locked-In Patient with ALS. N. Engl. J. Med. 375, 2060-2066 (2016).
[15] Moses, D. A. et al. Neuroprosthesis for Decoding Speech in a Paralyzed Person with Anarthria. N. Engl. J. Med. 385, 217-227 (2021).
[16] C. S. Mestais et al. WIMAGINE: Wireless 64-Channel ECOG Recording Implant for Long Term Clinical Applications. IEEE Trans. Neural Syst. Rehabil. Eng. 23, 10-21 (2015).
[17] Benabid, A. L. et al. An exoskeleton controlled by an epidural wireless brain-machine interface in a tetraplegic patient: a proof-of-concept demonstration. Lancet Neurol. (2019) doi: 10.1016/S1474-4422 (19) 30321-7.
[18] Seeber, M., Scherer, R., Wagner, J., Solis-Escalante, T. & Müller-Putz, G. R. EEG beta suppression and low gamma modulation are different elements of human upright walking. Front. Hum. Neurosci. 8, 485-485 (2014).
[19] Donati, A. R. C. et al. Long-Term Training with a Brain-Machine Interface-Based Gait Protocol Induces Partial Neurological Recovery in Paraplegic Patients. Sci. Rep. 6, 30383 (2016).
[20] McCrimmon, C. M. et al. Electrocorticographic Encoding of Human Gait in the Leg Primary Motor Cortex. Cereb. Cortex 28, 2752-2762 (2017).
[21] Selfslagh, A. et al. Non-invasive, Brain-controlled Functional Electrical Stimulation for Locomotion Rehabilitation in Individuals with Paraplegia. Sci. Rep. 9, 6782 (2019).
[22] Greiner, N. et al. Recruitment of upper-limb motoneurons with epidural electrical stimulation of the cervical spinal cord. Nat. Commun. 12, 435 (2021).
[23] Moly, A. et al. An adaptive closed-loop ECoG decoder for long-term and stable bimanual control of an exoskeleton by a tetraplegic. J. Neural Eng. 19, 026021 (2022).
[24] Cappellini, G., Ivanenko, Y. P., Dominici, N., Poppele, R. E. & Lacquaniti, F. Migration of motor pool activity in the spinal cord reflects body mechanics in human locomotion. J. Neurophysiol. 104, 3064-3073 (2010).
[25] Daly, J. J. et al. Development and testing of the Gait Assessment and Intervention Tool (G.A.I.T.): A measure of coordinated gait components. J. Neurosci. Methods 178, 334-339 (2009).
[26] Kathe, C. et al. The neurons that restore walking after paralysis. Nature 611, 540-547 (2022).
[27] Larzabal, C. et al. Long-term stability of the chronic epidural wireless recorder WIMAGINE in tetraplegic patients. J. Neural Eng. 18, (2021).
[28] Harkema, S. et al. Effect of epidural stimulation of the lumbosacral spinal cord on voluntary movement, standing, and assisted stepping after motor complete paraplegia: a case study. Lancet Lond. Engl. 377, 1938-1947 (2011).
[29] Rejc, E., Angeli, C. A., Atkinson, D. & Harkema, S. J. Motor recovery after activity-based training with spinal cord epidural stimulation in a chronic motor complete paraplegic. Sci. Rep. 7, 13476 (2017).
[30] Angeli, C. A. et al. Recovery of Over-Ground Walking after Chronic Motor Complete Spinal Cord Injury. N. Engl. J. Med. 379, 1244-1250 (2018).
[31] Gill, M. L. et al. Neuromodulation of lumbosacral spinal networks enables independent stepping after complete paraplegia. Nat. Med. 24, 1677-1682 (2018).
[32] Darrow, D. et al. Epidural Spinal Cord Stimulation Facilitates Immediate Restoration of Dormant Motor and Autonomic Supraspinal Pathways after Chronic Neurologically Complete Spinal Cord Injury. J. Neurotrauma 36, 2325-2336 (2019).
[33] Shokur, S. et al. Training with brain-machine interfaces, visuo-tactile feedback and assisted locomotion improves sensorimotor, visceral, and psychological signs in chronic paraplegic patients. PLOS ONE 13, e0206464 (2018).
[34] Biasiucci, A. et al. Brain-actuated functional electrical stimulation elicits lasting arm motor recovery after stroke. Nat. Commun. 9, 2421 (2018).
[35] Jovanovic, L. I. et al. Restoration of upper-limb function after chronic severe hemiplegia: A case report on the feasibility of a brain-computer interface controlled functional electrical stimulation therapy. Am. J. Phys. Med. Rehabil. Publish Ahead of Print, (2019).
[36] Nishimura, Y., Perlmutter, S. I., Eaton, R. W. & Fetz, E. E. Spike-timing-dependent plasticity in primate corticospinal connections induced during free behavior. Neuron 80, 1301-1309 (2013).
[37] Moorjani, S., Walvekar, S., Fetz, E. E. & Perlmutter, S. I. Movement-dependent electrical stimulation for volitional strengthening of cortical connections in behaving monkeys. Proc. Natl. Acad. Sci. 119, e2116321119 (2022).
[38] Guggenmos, D. J. et al. Restoration of function after brain damage using a neural prosthesis. Proc. Natl. Acad. Sci. 110, 21177-21182 (2013).
[39] Noga, B. R. & Guest, J. D. Combined neuromodulatory approaches in the central nervous system for treatment of spinal cord injury. Curr. Opin. Neurol. 34, 804-811 (2021).
[40] Barra, B. et al. Epidural electrical stimulation of the cervical dorsal roots restores voluntary upper limb control in paralyzed monkeys. Nat. Neurosci. 25, 924-934 (2022).
[41] Powell, M. P. et al. Epidural stimulation of the cervical spinal cord improves voluntary motor control in post-stroke upper limb paresis. medRxiv 2022.04.11.22273635 (2022) doi: 10.1101/2022.04.11.22273635.
[42] Knösche, T. R., & Haueisen, J. EEG/MEG source reconstruction: Textbook for electro- and magnetoencephalography. (2022).
[43] Gramfort, A. et al. MEG and EEG data analysis with MNE-Python. Front. Neurosci. 7, (2013).
[44] Tadel, F., Baillet, S., Mosher, J. C., Pantazis, D. & Leahy, R. M. Brainstorm: A User-Friendly Application for MEG/EEG Analysis. Comput. Intell. Neurosci. 2011, 879716 (2011).
[45] Auboiroux, V. et al. Space-Time-Frequency Multi-Sensor Analysis for Motor Cortex Localization Using Magnetoencephalography. Sensors 20, (2020).
[46] S. Robinet et al. A Low-Power 0.7μ Vᵣₘₛ 32-Channel Mixed-Signal Circuit for ECOG Recordings. IEEE J. Emerg. Sel. Top. Circuits Syst. 1, 451-460 (2011).
[47] Eliseyev, A. et al. Recursive Exponentially Weighted N-way Partial Least Squares Regression with Recursive-Validation of Hyper-Parameters in Brain-Computer Interface Applications. Sci. Rep. 7, 16281-16281 (2017).
[48] Sauter-Starace, F. et al. Long-Term Sheep Implantation of WIMAGINE., a Wireless 64-Channel Electrocorticogram Recorder. Front. Neurosci. 13, 847 (2019).
[49] Perry, J., Garrett, M., Gronley, J. K. & Mulroy, S. J. Classification of Walking Handicap in the Stroke Population. Stroke 26, 982-989 (1995).
[50] Tinetti, M. E., Franklin Williams, T. & Mayewski, R. Fall risk index for elderly patients based on number of chronic disabilities. Am. J. Med. 80, 429-434 (1986).
[51] Ermel, J. Von Fu. bis Kopf: Ganganalyse Teil 2: Anwendung in der Praxis. physiopraxis 4, 30-34 (2006).
[52] Mestais C S, Charvet G, Sauter-Starace F, Foerster M, Ratel D, Benabid A L. WIMAGINE: wireless 64-channel ECOG recording implant for long term clinical applications. IEEE Trans Neural Syst Rehabil Eng. 2015 January; 23 (1): 10-21. doi: 10.1109/TNSRE.2014.2333541. Epub 2014 Jun. 30. PMID: 25014960.
[53] Charvet G, Sauter-Starace F, Foerster M, Ratel D, Chabrol C, Porcherot J, Robinet S, Reverdy J, D'Errico R, Mestais C, Benabid AL. WIMAGINE (®): 64-channel ECOG recording implant for human applications. Annu Int Conf IEEE Eng Med Biol Soc. 2013; 2013:2756-9. doi: 10.1109/EMBC.2013.6610111. PMID: 24110298.
[54] Charvet G, Foerster M, Chatalic G, Michea A, Porcherot J, Bonnet S, Filipe S, Audebert P, Robinet S, Josselin V, Reverdy J, D'Errico R, Sauter F, Mestais C, Benabid AL. A wireless 64-channel ECoG recording electronic for implantable monitoring and BCI applications: WIMAGINE. Annu Int Conf IEEE Eng Med Biol Soc. 2012; 2012:783-6. doi: 10.1109/EMBC.2012.6346048. PMID: 23366009.
[55] Filipe S, Charvet G, Foerster M, Porcherot J, Bêche J F, Bonnet S, Audebert P, Régis G, Zongo B, Robinet S, Condemine C, Mestais C, Guillemaud R. A wireless multichannel EEG recording platform. Annu Int Conf IEEE Eng Med Biol Soc. 2011; 2011:6319-22. doi: 10.1109/IEMBS.2011.6091559. PMID: 22255783.
[56] Schirmer, C. M., Shils, J. L., Arle, J. E., Cosgrove, G. R., Dempsey, P. K., Tarlov, E., Kim, S., Martin, C. J., Feltz, C., Moul, M., & Magge, S. (2011). Heuristic map of myotomal innervation in humans using direct intraoperative nerve root stimulation, Journal of Neurosurgery: Spine SPI, 15 (1), 64-70. Retrieved Apr. 14, 2023, from https://doi.org/10.3171/2011.2.SPINE1068.
[57] Powell, M.P., Verma, N., Sorensen, E. et al. Epidural stimulation of the cervical spinal cord for post-stroke upper-limb paresis. Nat Med 29, 689-699 (2023). https://doi.org/10.1038/s41591-022-02202-6

### Supplementary Tables

SUPPLEMENTARY TABLE 1
WIMAGINE ® device specifications

| Analog front-end: CINESIC32 (x2) | | Wireless power supply | |
|---|---|---|---|
| Number of channels | 64 | Frequency | 13.56 MHz |
| Adjustable Gain | 1.5, 280, 990, 1370 (adjustable for each electrode) | Power | Adjustable up to 100 mW |
| Detection range | +/−1.3 mV (with Gain 990) | WIMAGINE Implant | |
| Bandwidth | 0.5-292 Hz | Norm | Class III DMIA: EN 45502-1 |
| Resolution | 12 bits - ADC architecture: SAR | Typical power requirement | 25 mA/3.3 V |
| Noise level | <2.5 μV RMS (with Gain 990 on BW 0.5-292 Hz) | | |
| Sampling rate | 586 Hz or 1 kHz adjustable | Electrode open area | 2.0 mm in diameter |
| Microcontrolers and Sensors | | Electrode spacing | 4.0 mm and 4.5 mm |
| Microcontroler | MSP430F2618-EP (Texas Instrument) | | |
| Sensors | Accelerometer, temperature, power consumption, | | |
| | voltage | | |
| UHF link | | | |
| Component | Transciever Microsemi ZL70102 (Zarlink) | | |
| Frequency | 402-405 MHz | | |
| Data rate-range | 250 kb/s-5 cm (2FSK) | | |

SUPPLEMENTARY TABLE 2
Epidural electrical stimulation system specifications

| | | Paddle lead Specify ® SureScan ® MRI 5-6-5 | |
|---|---|---|---|
| ACTIVA RC 37612, Medtronic | | Lead Kit 977C190, Medtronic | |
| Number of channels | 16 | Electrode dimensions | 1.5 mm × 4.0 mm |
| Stimulation amplitude | 0-25.5 mA (0.1 mA | Paddle dimensions | Adjustable up to |
| | resolution) | | 100 mW |
| Stimulation frequency | 0.1-500 Hz | Spacing | 1 mm × 4 mm |
| (with modified firmware) | | | |
| Pulse width | 60-450 μs | Paddle length | 64.2 mm |

SUPPLEMENTARY TABLE 3
Observation gait analysis

| OBSERVATIONAL GAIT ANALYSIS | | | | |
|---|---|---|---|---|
| Parameter | Criterion | L | R | References |
| Head position | Looks mostly straight ahead | 2 | | Translated into English, added one point |
| | Alternates with look straight and to the floor | 1 | | about alternating head position |
| | Looks mostly to the floor | 0 | | |
| Trunk Posture | Upright position | 2 | | G.A.I.T. |
| (Flexion-Extension) | Trunk flexes or extends slightly during stepping | 1 | | Simplified “until 30°” to “slightly” and |
| | Trunk flexes or extends more than 30° | 0 | | use it during all gait phases |
| Trunk Posture (Lateral | Upright position | 2 | | G.A.I.T. |
| Lean) | Trunk leans slightly to the right or left | 1 | | Simplified “until 30°” to “slightly” and |
| | Trunk leans to the right or left more than 30° | 0 | | use it during all gait phases |
| Pelvic Position (Stance | Normal (no Trendelenberg sign) | 2 | 2 | G.A.I.T. |
| Phase) | Mild pelvic drop on contralateral side. | 1 | 1 | |
| | Severe or abrupt pelvic drop on contralateral side. | 0 | 0 | |
| Pelvic Position (Swing | Normal (relatively level pelvis or slightly lower on swing side). | 2 | 2 | G.A.I.T. |
| Phase) | Mild hip hiking. | 1 | 1 | |
| | Moderate to severe hip hiking. | 0 | 0 | |
| Weight shift | Shifts weight to stance limb. only uses parallel bars for security | 2 | 2 | SCI-FAI |
| | Leans slightly on arms and parallel bars to weight shift | 1 | 1 | Added “only uses Parallel bars for |
| | Leans heavily on arms and parallel bars to weight shift | 0 | 0 | security”plus another point to distinguish |
| | | | | the use of arms |
| Step Continuity | Steps appear continuous | 1 | 1 | Tinetti Test |
| | Stopping or discontinuity between steps | 0 | 0 | |
| Step rhythm (relative | At heel strike of stance limb, the swing limb: | 2 | 2 | SCI-FAI |
| time needed to | Begins to advance in <1 second or | 1 | 1 | |
| advance swing limb) | Requires 1-3 seconds to begin advancing or | 0 | 0 | |
| | Requires >3 seconds to begin advancing | | | |
| Step height | Toe clears floor throughout swing phase or | 2 | 2 | SCI-FAI |
| | Toe drags at initiation of swing phase only or | 1 | 1 | |
| | Toe drags throughout swing phase | 0 | 0 | |
| Step Length | Swing heel placed forward of stance toe or | 2 | 2 | SCI-FAI |
| | Swing toe placed forward of stance toe or | 1 | 1 | |
| | Swing toe placed rearward of stance toe | 0 | 0\| | |
| Step Symmetry | Right and left step length appear equal | 1 | | Tinetti Test |
| | Right and left step length not equal (estimate) | 0 | | |
| Step width | Swing foot clears stance foot on limb advancement | 1 | 1 | SCI-FAI |
| | Stance foot obstructs swing foot on limb advancement | 0 | 0 | |
| | Final foot placement does not obstruct swing limb | 1 | 1 | |
| | Final foot placement obstructs swing limb | 0 | 0 | |
| Foot contact | Heel contacts floor before forefoot or | 2 | 2 | SCI-FAI |
| | Foot flat first contact with floor | 1 | 1 | added another point to distinguish foot |
| | Forefoot contacts floor before heel | 0 | 0 | flat and forefoot contact |

SUPPLEMENTARY TABLE 4
Psychosocial Impact of Assistive Devices Scale
(PIADS) questionnaire during the home use phase

| | | | | | | | | |
|---|---|---|---|---|---|---|---|---|
| Decreases | −3 | −2 | −1 | 0 | 1 | 2 | 3 | Increase |

| | | | | | |
|---|---|---|---|---|---|
| 1) competence | | | x | | |
| 2) happiness | | | x | | |
| 3) independence | | x | | | |
| 4) adequacy | | x | | | |
| 5) confusion | | x | | | |
| 6) efficiency | | | x | | |
| 7) self-esteem | | | | x | |
| 8) productivity | | | | x | |
| 9) security | | | | | x |
| 10) frustration | x | | | | |
| 11) usefulness | | | x | | |
| 12) self-confidence | | | | x | |
| 13) expertise | | | x | | |
| 14) skillfulness | | | | | x |
| 15) well-being | | | x | | |
| 16) capability | | | | x | |
| 17) quality of life | | | | x | |
| 18) performance | | | x | | |
| 19) sense of power | | | x | | |
| 20) sense of control | | | | x | |
| 21) embarrassment | x | | | | |
| 22) willingness to take chances | | | x | | |
| 23) ability to participate | | | | | x |
| 24) eagerness to try new thing | | | | x | |
| 25) ability to adapt to the activities | | | | x | |
| of daily living | | | | | |
| 26) ability to take advantage | | | | x | |

1. A neuromodulation/neurostimulation system for restoring a motor function of upper and/or lower limbs and facilitating neurological recovery in a patient with spinal cord injury and/or other neurological disorders, said system comprising:
a processing unit, which is configured and adapted provide stimulation commands;
an implantable or external stimulation unit, which is configured and adapted to deliver neuromodulation therapy to dorsal roots of a spinal cord of said patient;
at least one implantable or external pulse generator, operatively connected to the stimulation unit; and
an implantable neurosensor including electronic components that are configured and adapted to detect electrocorticographic signals from a sensorimotor cortex of said patient, denoting a motor intention of said patient,
wherein the processing unit, the stimulation unit, and the implantable neurosensor are operatively connected to define a brain-spinal interface, and
wherein the processing unit is configured and adapted to:
collect and decode the electrocorticographic signals from the sensorimotor cortex into a corresponding motor intention of said patient;
convert the decoded motor intentions into corresponding stimulation commands, and
forward the stimulation command to the at least one pulse generator.

2. The neuromodulation/neurostimulation system according to claim 1,
wherein the processing unit includes software means that are configured and adapted to decode one or more brain signals from the received electrocorticographic signals.

3. The neuromodulation/neurostimulation system according to claim 1,
wherein the processing unit, the stimulation unit, and the implantable neurosensor are operatively connected through a wireless connection.

4. The neuromodulation/neurostimulation system according to claim 1,
wherein the stimulation unit is configured and adapted to deliver neuromodulation therapy to a lumbosacral region of the spinal cord of said patient to restore a motor function of the lower limbs.

5. The neuromodulation/neurostimulation system according to claim 1,
wherein the stimulation unit is configured and adapted to deliver neuromodulation therapy to a cervical region of the spinal cord of said patient to restore a motor function of the upper limbs.

6. The neuromodulation/neurostimulation system according to claim 1,
wherein the implantable neurosensor is configured to be located over the dura mater above a fraction of the sensorimotor cortex.

7. The neuromodulation/neurostimulation system according to claim 6,
wherein the implantable neurosensor includes at least one cortical implant comprising 64 electrodes that are configured to be epidurally implanted over the dura mater above a fraction of the sensorimotor cortex.

8. The neuromodulation/neurostimulation system according to claim 7,
wherein said 64 electrodes of each cortical implant are arranged according to an 8-by-8 grid.

9. The neuromodulation/neurostimulation system according to claim 1,
wherein the electronic components of the implantable neurosensor are provided within a case having the same thickness as a skull of the patient.

10. The neuromodulation/neurostimulation system according to claim 9,
wherein the case is made of Titanium.

11. The neuromodulation/neurostimulation system according to claim 1, wherein said neuromodulation/neurostimulation system further includes:
a first external, high frequency antenna, which is configured and adapted to power the implanted components of the neuromodulation/neurostimulation system through inductive coupling, and
a second external, ultra-high frequency antenna, which is configured and adapted for real-time transfer of the detected electrocorticographic signals to the processing unit.

12. The neuromodulation/neurostimulation system according to claim 11,
wherein the first external, high frequency antenna and the second external, ultra-high frequency antenna are embedded within a wearable device.

13. The neuromodulation/neurostimulation system according to claim 11,
wherein the first external, high frequency antenna and the second external, ultra-high frequency antenna are operatively connected to a base station, said base station being configured and adapted to perform at least:
data conditioning;
power generation, and/or
synchronization with other devices to allow interfacing with the processing unit.

14. The neuromodulation/neurostimulation system according to claim 1,
wherein the processing unit is embedded within a wearable device.

15. The neuromodulation/neurostimulation system according to claim 1,
wherein the pulse generator is implemented with at least one communication module that is configured and adapted to implement real-time adjustment over the stimulation target and timing of delivered neuromodulation therapy with a latency of about 100 ms.

16. The neuromodulation/neurostimulation system according to claim 1,
wherein the stimulation unit includes an implantable paddle lead comprising an array of 16 electrodes configured to be epidurally implanted at a target location of the spinal cord of said patient.

17. The neuromodulation/neurostimulation system according to claim 4,
wherein said motor function of the lower limbs includes control over independent joint movements of the hip, knee and/or ankles bilaterally, standing, walking on a plain terrain, walking on a complex terrain, and/or stairs climbing.

18. The neuromodulation/neurostimulation system according to claim 5,
wherein said motor function of the upper limbs includes independent control over different joint movements.

19. The neuromodulation/neurostimulation system according to claim 1,
wherein the stimulation unit is configured to deliver neuromodulation therapy according to predefined stimulation parameters, wherein said predefined stimulation parameters include at least stimulation frequency, stimulation amplitude, pulse width, location of one or multiple cathodes, and/or location of one or multiple anodes.

20. A walking-assistance device for use with the neuromodulation/neurostimulation system according to claim 1, said device comprising:
at least one charging module connected to a power inlet, and
a user interface allowing a user to trigger, stop and/or adjust delivered stimulation, wherein the processing unit is embedded within the walking-assistance device.

21. The walking-assistance device according to claim 20,
wherein said walking-assistance device further comprises one or more of the following components:
a camera for markerless tracking of body posture;
an Inertial Measurement Unit base station;
one or more manual clickers, and/or
one or more hybrid clickers.
